SEQUENCE LISTING (37 sequences)

SEQ ID NO: 1 - protein, 154 aa, Bos indicus
MGLSDGEWQLVLNAWGKVEADVAGHGQEVLIRLFTGHPETLEKFDKFKHLKTEAEMKASEDLKKHGNTVLTALGGILKKKGHHEAEVKHLAESHANKHKIPVKYLEFISDAIIHVLHAKHPSDFGADAQAAMSKALELFRNDMAAQYKVLGFHG

SEQ ID NO: 2 - protein, 154 aa, Bos mutus; identical to SEQ ID NO: 1

SEQ ID NO: 3 - protein, 154 aa, Bos indicus x Bos taurus; identical to SEQ ID NO: 1

SEQ ID NO: 4 - protein, 154 aa, Bos taurus; identical to SEQ ID NO: 1

SEQ ID NO: 5 - protein, 154 aa, Bos mutus; identical to SEQ ID NO: 1

SEQ ID NO: 6 - protein, 154 aa, Bison bison; identical to SEQ ID NO: 1

SEQ ID NO: 7 - protein, 154 aa, Ovis aries
MGLSDGEWQLVLNAWGKVEADVAGHGQEVLIRLFTGHPETLEKFDKFKHLKTEAEMKASEDLKKHGNTVLTALGGILKKKGHHEAEVKHLAESHANKHKIPVKYLEFISDAIIHVLHAKHPSDFGADAQGAMSKALELFRNDMAAQYKVLGFQG

SEQ ID NO: 8 - protein, 154 aa, Rangifer tarandus; identical to SEQ ID NO: 7

SEQ ID NO: 9 - protein, 154 aa, Bubalus bubalis
MGLSDGEWQLVLNAWGKVETDVAGHGQEVLIRLFTGHPETLEKFDKFKHLKTEAEMKASEDLKKHGNTVLTALGGILKKKGHHEAEVKHLAESHANKHKIPVKYLEFISDAIIHVLHDKHPSDFGADAQAAMSKALELFRNEMAAQYKVLGFHG

SEQ ID NO: 10 - protein, 154 aa, Cervus elaphus
MGLSDGEWQLVLNAWGKVEADVAGHGQEVLIRLFTGHPETLEKFDKFKHLKTEAEMKASEDLKKHGNTVLTALGGILKKKGHHEAEVKHLAESHANKHKIPVKYLEFISDAIIHVLHAKHPSNFGADAQGAMSKALELFRNDMAAQYKVLGFQG

SEQ ID NO: 11 - protein, 154 aa, Capra hircus
MGLSDGEWTLVLNAWGKVEADVAGHGQEVLIRLFTGHPETLEKFDKFKHLKTGAEMKASEDLKKHGNTVLTALGGILKKKGHHEAEVKHLAESHANKHKIPVKYLEFISDAIIHVLHAKHPSDFGADAQGAMSKALELFRNDMAAQYKVLGFQG

SEQ ID NO: 12 - protein, 154 aa, Inia geoffrensis
MGLSDGEWQLVLNIWGKVEADLAGHGQDVLIRLFKGHPETLEKFDKFKHLKTEAEMKASEDLKKHGNTVLTALGGILKKKGHHEAELKPLAQSHATKHKIPIKYLEFISEAIIHVLHSRHPGDFGADAQAAMNKALELFRKDIAAKYKELGFHG

SEQ ID NO: 13 - protein, 154 aa, Equus caballus
MGLSDGEWQQVLNVWGKVEADIAGHGQEVLIRLFTGHPETLEKFDKFKHLKTEAEMKASEDLKKHGTVVLTALGGILKKKGHHEAELKPLAQSHATKHKIPIKYLEFISDAIIHVLHSKHPGDFGADAQGAMTKALELFRNDIAAKYKELGFQG

SEQ ID NO: 14 - protein, 154 aa, Oryctolagus cuniculus
MGLSDAEWQLVLNVWGKVEADLAGHGQEVLIRLFHTHPETLEKFDKFKHLKSEDEMKASEDLKKHGNTVLTALGAILKKKGHHEAEIKPLAQSHATKHKIPVKYLEFISEAIIHVLHSKHPGDFGADAQAAMSKALELFRNDIAAQYKELGFQG

SEQ ID NO: 15 - protein, 154 aa, Sus scrofa
MGLSDGEWQLVLNVWGKVEADVAGHGQEVLIRLFKGHPETLEKFDKFKHLKSEDEMKASEDLKKHGNTVLTALGGILKKKGHHEAELTPLAQSHATKHKIPVKYLEFISEAIIQVLQSKHPGDFGADAQGAMSKALELFRNDMAAKYKELGFQG

SEQ ID NO: 16 - protein, 154 aa, Balaenoptera acutorostrata
MVLSDAEWHLVLNIWAKVEADVAGHGQDILIRLFKGHPETLEKFDKFKHLKTEAEMKASEDLKKHGNTVLTALGGILKKKGHHEAELKPLAQSHATKHKIPIKYLEFISDAIIHVLHSRHPAEFGADAQAAMNKALELFRKDIAAKYKELGFQG

SEQ ID NO: 17 - protein, 154 aa, Homo sapiens
MGLSDGEWQLVLNVWGKVEADIPGHGQEVLIRLFKGHPETLEKFDKFKHLKSEDEMKASEDLKKHGATVLTALGGILKKKGHHEAEIKPLAQSHATKHKIPVKYLEFISECIIQVLQSKHPGDFGADAQGAMNKALELFRKDMASNYKELGFQG

SEQ ID NO: 18 - protein, 154 aa, Macropus rufus
MGLSDGEWQLVLNIWGKVETDEGGHGKDVLIRLFKGHPETLEKFDKFKHLKSEDEMKASEDLKKHGITVLTALGNILKKKGHHEAELKPLAQSHATKHKIPVQFLEFISDAIIQVIQSKHAGNFGADAQAAMKKALELFRHDMAAKYKEFGFQG

SEQ ID NO: 19 - protein, 154 aa, Bambusicola thoracica
MGLSDQEWQQVLAIWGKVEADIAGHGHEVLMRLFRDHPETLDRFDKFKGLKTPDQMKGSEDLKKHGATVLTQLGKILKQKGNHEAELKPLAQTHATKHKIPVKYLEFISEVIIKVIAEKHAADFGADSQAAMKKALELFRNDMASKYKEFGFQG

SEQ ID NO: 20 - protein, 154 aa, Odontophorus gujanensis
MGLSDQEWQQVLSIWGKVEADIAGHGHEVLMRLFHDHPETLDRFEKFKGLKTPDQMKGSEDLKKHGATVLTQLGKILKQKGNHESELKPLAQTHATKHKIPVKYLEFISEVIIKVIAEKHAADFGADSQAAMKKALELFRNDMAAKYKEFGFQG

SEQ ID NO: 21 - protein, 154 aa, Meleagris gallopavo
MGLSDQEWQQVLTIWGKVEADIAGHGHEVLMRLFHDHPETLDRFDKFKGLKTPDQMKGSEDLKKHGATVLTQLGKILKQKGNHESELKPLAQTHATKHKIPVKYLEFISEVIIKVIAEKHAADFGADSQAAMKKALELFRNDMASKYKEFGFQG

SEQ ID NO: 22 - protein, 154 aa, Gallus gallus; identical to SEQ ID NO: 21

SEQ ID NO: 23 - protein, 154 aa, Anseranas semipalmata
MGLSDQEWQHVLTIWGKVEADLAGHGHAVLIRLFQDHPETLDRFEKFKGLKTPDQMKGSEDLKKHGVTVLTQLGKILKQKGNHEAELKPLAQTHATKHKIPVKYLEFISEVIIKVIAEKHSADFGADSQAAMKKALELFRNDMASKYKEFGFQG

SEQ ID NO: 24 - protein, 154 aa, Anas platyrhynchos
MGLSDQEWQQVLTIWGKVEADLAGHGHAVLMRLFQDHPETLDRFEKFKGLKTPDQMKGSEDLKKHGVTVLTQLGKILKQKGNHEAELKPLAQTHATKHKIPVKYLEFISEVIIKVIAEKHSADFGADSQAAMKKALELFRNDMASKYKEFGFQG

SEQ ID NO: 25 - protein, 154 aa, Anas poecilorhyncha; identical to SEQ ID NO: 24

SEQ ID NO: 26 - protein, 154 aa, Struthio camelus
MGLSDQEWQQVLTIWGKVESDIAGHGHAILMRLFQDHPETLDRFEKFKGLTTPEQMKASEELKKHGVTVLTQLGKILKQKGKHEAELKPLAQTHATKHKIPVKYLEFISEVIIKVIAEKHSADFGADSQAAMKKALELFRNDMASKYKEFGFQG

SEQ ID NO: 27 - protein, 147 aa, Thunnus albacares
MADFDAVLKCWGPVEADYTTMGGLVLTRLFKEHPETQKLFPKFAGIAQADIAGNAAISAHGATVLKKLGELLKAKGSHAAILKPLANSHATKHKIPINNFKLISEVLVKVMHEKAGLDAGGQTALRNVMGIIIADLEANYKELGFSG

SEQ ID NO: 28 - protein, 147 aa, Schizothorax labiatus
MADHDLVLKCWGAIEADFTGHGGDVLNRLFKEHPETLKLFPKFVSIPPSDLVGNAAVAAHGATVLTKLGELLKARGDHAALLKPLATTHANKHKIALNNFKLITEVLVKVMAEKAGLDAAGQSAFKRVMEAVIGDIDTYYKEIGFAG

SEQ ID NO: 29 - protein, 147 aa, Auxis rochei
MADFDAVLKCWGPVEADFNTVGGMVLARLFKDHPDTQKLFPKFAGIAAGDLAGNAAVAAHGGTVLKKLGELLKAKGNHAAIIKPLANSHATKHKIPINNFKLITEALVHVMQEKAGLDAAGQTALRNVMGIVIADLEANYKELGFTG

SEQ ID NO: 30 - protein, 146 aa, Katsuwonus pelamis
MADLDAVLKCWGAVEADFNTVGGLVLARLFKDHPETQKLFPKFAGITGDIAGNAAVAAHGATVLKKLGELLKAKGNHAAIIKPLANSHAKQHKIPINNFKLITEALAHVLHEKAGLDAAGQTALRNVMGIVIADLEANYKELGFTG

SEQ ID NO: 31 - protein, 144 aa, Gadus morhua
MADYDLVLRCWGPVEADYNTHGGLVLTRLFTEHPDTQKLFPKLAGVGELAASVAVASHGATVLKKLGELLKTRGDHAALLKPLATSHANVHKIPISNFKLITEVIAKHMAEKAGLDAAGQEALREVMSVVIADMDATYKELGFS

SEQ ID NO: 32 - protein, 147 aa, Scomber japonicus
MADFDAVLKFWGPVEADYDKIGNMVLTRLFTEHPDTQKLFPKFAGIGLGDMAGNAAISAHGATVLKKLAEVLKAKGNHAGIIKPLANSHATKHKIAINNFKLITEIIVKVMQEKAGLDAGGQTALRNVMGVFIADMDANYKELGFSG

SEQ ID NO: 33 - protein, 141 aa, Anguilla anguilla
FELVLKAWKPIEADLKGNGGVVLTRLFQEHPETQQLFPKFAAIAPGDLAGNAAISEHGCTVLTKLGDLLHAKGNHADILKPLAKTHATQHKIKLQNFQLITEVIVKLMGEKGVDAAGQEAVRKVMLAVIGDIDNFYKVLGF

SEQ ID NO: 34 - protein, 147 aa, Salmo salar
MANYDMVLQCWEPVEADYNNHGGLVLSRLFAEHPETLTLFPKFAGIAAGDLSGNAAVAAHGATVLRKLGELLNARGDHAATLKSLATTHANKHKIPLKNFTLITNIICKVMGEKAGLDEAGQEALRQVMGVIIADINVTYMELGFAG

SEQ ID NO: 35 - protein, 147 aa, Thunnus thynnus
MADFDAVLKCWGPVEADYTTIGGLVLTRLFKEHPETQKLFPKFAGIAQADIAGNAAVSAHGATVLKKLGELLKAKGSHAAILKPLANSHATKHKIPINNFKLISEVLVKVMHEKAGLDAGGQTALRNVMGIIIADLEANYKELGFSG

SEQ ID NO: 36 - protein, 6 aa, Artificial Sequence
  6X-His
HHHHHH

SEQ ID NO: 37 - protein, 8 aa, Artificial Sequence
  FLAG-tag
DYKDDDDK

(19) United States
(12) Patent Application Publication
(10) Pub. No.: US 2024/0200088 A1
(43) Pub. Date: Jun. 20, 2024

(54) Transgenic Plants Comprising Myoglobin and Methods for Producing Myoglobin in Transgenic Plants

(71) Applicant: KYOMEI LTD., London (GB)

(72) Inventors: Kyoko MORIMOTO, Berkshire (GB); Yisroel Meir WACHS, Philadelphia, PA (US)

(21) Appl. No.: 18/286,484

(22) PCT Filed: Apr. 13, 2022
(86) PCT No.: PCT/US2022/024616

§ 371 Date: Oct. 11, 2023

Related U.S. Application Data
(60) Provisional application No. 63/174,484, filed on Apr. 13, 2021.

(51) Int. Cl. C12N 15/82; C07K 14/805; C12N 15/52

(52) U.S. Cl. CPC C12N 15/8257; C07K 14/805; C12N 15/52

(57) ABSTRACT

This disclosure provides methods and materials for producing a transgenic plant expressing a myoglobin gene, producing myoglobin protein in the transgenic plant, and isolating the myoglobin protein from the transgenic plants.

### CROSS REFERENCE TO RELATED APPLICATIONS

This application claims priority to U.S. Provisional Application No. 63/174,484, filed Apr. 13, 2021, the disclosure of which is incorporated by reference in its entirety.

### STATEMENT REGARDING SEQUENCE LISTING

A computer readable form of the Sequence Listing is filed with this application by electronic submission and is incorporated into this application by reference in its entirety. The Sequence Listing is contained in the ASCII text file created on Apr. 13, 2022, having the file name “21-0399-WO_Sequence-Listing_ST25.txt” and is 48 kb in size.

### BACKGROUND

### Technical Field

The invention disclosed herein relates generally to the field of genetic engineering. Particularly, the invention disclosed herein provides methods and materials for producing a transgenic plant expressing a myoglobin gene, producing myoglobin protein in the transgenic plant, and isolating the myoglobin protein from the transgenic plants.

### Description of Related Art

Livestock farming has an enormous environmental impact and contributes to land and water degradation, biodiversity loss, and deforestation. Demand for animal meat alternatives has grown and will continue to rise, with the global meat substitutes sector valued at over $20 billion, and projected grow to over $24 billion in the next few years. There is a growing need for alternative ways to produce animal meat proteins in an efficient, sustainable, and scalable manner. Employing a plant-based protein production system is an emerging field that has seen some success in the pharmaceutical industry for vaccine production.

### SUMMARY

It is against the above background that the present disclosure provides certain advantages and advancements over the prior art. Although this invention disclosed herein is not limited to specific advantages or functionality, the invention disclosed herein provides methods and materials for producing a transgenic plant expressing a myoglobin gene, producing myoglobin protein in the transgenic plant, and isolating the myoglobin protein from the transgenic plants.

These and other features and advantages of the present invention will be more fully understood from the following detailed description of the invention taken together with the accompanying claims. It is noted that the scope of the claims is defined by the recitations therein and not by the specific discussion of features and advantages set forth in the present description.

Disclosed herein are methods and materials to produce recombinant animal meat proteins in plants that is more sustainable and cost efficient compared to conventional methods (e.g. yeast and bacterial cell cultures), as plants obtain energy from sunlight by photosynthesis and can be planted in open fields. As demonstrated herein, stable chloroplast transformation in plants provides for commercial scale manufacturing of myoglobin meat protein in transgenic plants. Growing transformed plants to produce animal meat has few, if any, adverse impacts on the environment, and results in a net positive impact on CO₂ emissions, even at commercial production levels. There are unique advantages of chloroplast transformation technologies where the recombinant genes of interest are integrated into a targeted site of the chloroplast genome by homologous recombination. For example, non-limiting examples of chloroplast transformation can include: a) higher expression of foreign genes because of multiple copies (1,000-50,000 copies) of the genes due to the multi-copy of chloroplast DNA (100-250 copies) per chloroplast and multi-copy of chloroplasts in the cells; b) higher accumulation of proteins (˜70% of total soluble proteins) because of the compartmentalization of the proteins; c) simultaneous expression of several genes under the single promoter as chloroplast has a prokaryotic gene expression system; d) little instability of foreign genes (e.g. silencing, positional effect); and e) low risk of gene dispersal in the environment because of the single-parent inheritance of chloroplast genome.

In an aspect, this disclosure provides a transgenic plant, wherein the transgenic plant comprises at least one chloroplast with one or more recombinant nucleic acid sequences expressing a myoglobin gene encoding a myoglobin protein. In some embodiments, the transgenic plant comprises the one or more recombinant nucleic acid sequences integrated into the chloroplast DNA of the transgenic plant. In certain embodiments, the transgenic plant comprises the one or more recombinant nucleic acid sequences stably integrated into the chloroplast DNA of the transgenic plant. In some embodiments, the transgenic plant comprises at least about 10 copies, at least about 100 copies, at least about 1,000 copies, at least about 5,000 copies, at least about 10,000 copies, at least about 20,000 copies, at least about 30,000 copies, at least about 40,000 copies, or at least about 50,000 copies of the one or more recombinant nucleic acid sequences.

In certain embodiments of the transgenic plant, the one or more recombinant nucleic acid sequences further comprises: (a) one or more selectable markers, wherein the one or more selectable markers are optionally removable; (b) one or more genes encoding one or more enzymes in the heme biosynthesis pathway; and/or (c) one or more targeting sequences for homologous recombination in the host transgenic plant chloroplast DNA.

In some embodiments, the transgenic plant is a stable, homoplasmic transformant. In some embodiments, the transgenic plant is a stable heteroplasmic transformant.

In certain embodiments of the transgenic plant, the myoglobin gene is a bovine myoglobin gene (for example, bison, buffalo, cow, goat, sheep, or yak), an avian myoglobin gene (for example, chicken, duck, goose, guinea fowl, quail, pigeon, or turkey), a suine myoglobin gene (for example, boar or pig), or a fish myoglobin (for example, tuna, salmon, or eel). In some embodiments, the myoglobin gene is selected from any of the genes of Table 1 and/or wherein the myoglobin gene encodes a myoglobin protein selected from SEQ ID NO's 1-35.

In certain embodiments of the transgenic plant, the myoglobin gene comprises a codon-optimized myoglobin gene, and wherein the codon-optimized myoglobin gene is codon-optimized for expression in the transgenic plant. In some embodiments, the myoglobin gene is operably linked to at least one promoter.

In certain embodiments, the transgenic plant is a grass (for example, a barely, a corn, a maize, an oat, a silvergrass, a sugarcane, a rice, a rye, or a wheat), a legume (for example, an alfalfa, a bean, a chickpea, a clover, a lentil, a pea, or a peanut), a nightshade (for example, an eggplant, a pepper, a potato, a tobacco, or a tomato), an aster (for example, a lettuce, a chamomile, an artichoke, an endive, a lavender, a cotton, a sunflower), or an alga, a moss, or a liverwort. In some embodiments, the transgenic plant is a legume, and the legume is a soybean (Glycine max), a pea (Pisum satiyum), or a lupine (Lupinus mutabilis). In some embodiments, the transgenic plant is an aster, and the aster is a lettuce plant (i.e., a Lachica species). In certain embodiments, the transgenic plant is a nightshade, and the nightshade is a tobacco plant (i.e., a Nicotiana species). In some embodiments, the tobacco plant is a nicotine-free tobacco plant. In some embodiments, the tobacco plant is a wild-type tobacco plant.

In some embodiments, the transgenic plant comprises a knock-down or knock-out of one or more genes encoding magnesium chelatase enzymes. In certain embodiments, the transgenic plants as described herein comprise modified, mutated, and/or knockouts or knockdowns of one or more genes encoding magnesium chelatase enzymes selected from the genes of Table 3 and/or Table 4.

In some embodiments of the transgenic plant, the myoglobin protein comprises at least about 0.1%, at least about 1.0%, at least about 10%, at least about 20%, at least about 30%, at least about 40%, at least about 50%, at least about 60%, at least about 70%, at least about 80%, or at least about 90% of the total soluble protein from the transgenic plant.

In some embodiments, the disclosure provides a method of producing a myoglobin protein, comprising growing the transgenic plant as disclosed herein and isolating the myoglobin protein from the transgenic plant.

In another aspect, this disclosure provides a method of producing a myoglobin protein in a transgenic plant, wherein the method comprises: (a) growing the transgenic plant, wherein the transgenic plant comprises at least one chloroplast with one or more recombinant nucleic acid sequences expressing a myoglobin gene encoding the myoglobin protein, and (b) isolating the myoglobin protein from the transgenic plant.

In some embodiments of the method, the one or more recombinant nucleic acid sequences is integrated into the chloroplast DNA of the transgenic plant. In some embodiments of the method, the one or more recombinant nucleic acid sequences is stably integrated into the chloroplast DNA of the transgenic plant. In certain embodiments, the transgenic plant comprises at least about 10 copies, at least about 100 copies, at least about 1,000 copies, at least about 5,000 copies, at least about 10,000 copies, at least about 20,000 copies, at least about 30,000 copies, at least about 40,000 copies, or at least about 50,000 copies of the one or more recombinant nucleic acid sequences.

In some embodiments of the method, the one or more recombinant nucleic acid sequences further comprises: (a) one or more selectable markers, wherein the one or more selectable markers are optionally removable; (b) one or more genes encoding one or more enzymes in the heme biosynthesis pathway; and/or (c) one or more targeting sequences for homologous recombination in the host transgenic plant chloroplast DNA.

In some embodiments of the method, the transgenic plant is a stable, homoplasmic transformant. In some embodiments, the transgenic plant is a stable heteroplasmic transformant.

In some embodiments of the method, the myoglobin gene is a bovine myoglobin gene (for example, bison, buffalo, cow, goat, sheep, or yak), an avian myoglobin gene (for example, chicken, duck, goose, guinea fowl, quail, pigeon, or turkey), a suine myoglobin gene (for example, boar or pig), or a fish myoglobin (for example, tuna, salmon, or eel). In certain embodiments, the myoglobin gene is selected from any of the genes of Table 1 and/or wherein the myoglobin gene encodes a myoglobin protein selected from SEQ ID NO's 1-35. In some embodiments of the method, the myoglobin gene comprises a codon-optimized myoglobin gene, wherein the codon-optimized myoglobin gene is codon-optimized for expression in the transgenic plant. In some embodiments of the method, the myoglobin gene is operably linked to at least one promoter.

In some embodiments of the method, the transgenic plant is a grass (for example, a barely, a corn, a maize, an oat, a silvergrass, a sugarcane, a rice, a rye, or a wheat), a legume (for example, an alfalfa, a bean, a chickpea, a clover, a lentil, a pea, or a peanut), a nightshade (for example, an eggplant, a pepper, a potato, a tobacco, or a tomato), an aster (for example, a lettuce, a chamomile, an artichoke, an endive, a lavender, a cotton, a sunflower), or an alga, a moss, or a liverwort. In certain embodiments, the transgenic plant is a legume, and the legume is a soybean (Glycine max), a pea (Pisum satiyum), or a lupine (Lupinus mutabilis). In some embodiments, the transgenic plant is an aster, and the aster is a lettuce plant (i.e., a Lactica species). In some embodiments, the transgenic plant is a nightshade, and the nightshade is a tobacco plant (i.e., a Nicotiana species). In some embodiments, the tobacco plant is a nicotine-free tobacco plant. In certain embodiments, the tobacco plant is a wild-type tobacco plant.

In some embodiments, the transgenic plant comprises a knock-down or knock-out of one or more magnesium chelatase enzymes.

In some embodiments of the method, the myoglobin protein comprises at least about 0.1%, at least about 1.0%, at least about 10%, at least about 20%, at least about 30%, at least about 40%, at least about 50%, at least about 60%, at least about 70%, at least about 80%, or at least about 90% of the total soluble protein from the transgenic plant.

In another aspect, this disclosure provides a recombinant nucleic acid sequence comprising a myoglobin gene encoding a myoglobin protein, wherein the myoglobin gene is operably linked to at least one promoter. In some embodiments, the recombinant nucleic acid sequence further comprises: (a) one or more selectable markers, wherein the one or more selectable markers are optionally removable; (b) one or more genes encoding one or more enzymes in the heme biosynthesis pathway; and/or (c) one or more targeting sequences for homologous recombination in the host transgenic plant chloroplast DNA.

In some embodiments of the recombinant nucleic acid sequence, the myoglobin gene is a bovine myoglobin gene (for example, bison, buffalo, cow, goat, sheep, or yak), an avian myoglobin gene (for example, chicken, duck, goose, guinea fowl, quail, pigeon, or turkey), a suine myoglobin gene (for example, boar or pig), or a fish myoglobin (for example, tuna, salmon, or eel). In certain embodiments, the myoglobin gene is selected from any of the genes of Table I and/or wherein the myoglobin gene encodes a myoglobin protein selected from SEQ ID NO's 1-35. In some embodiments, the myoglobin gene comprises a codon-optimized myoglobin gene, wherein the codon-optimized myoglobin gene is codon-optimized for expression in a transgenic plant.

In some embodiments this disclosure provides a transgenic plant comprising the recombinant nucleic acid sequence as disclosed herein. In certain embodiments, the recombinant nucleic acid sequence is integrated into the chloroplast DNA of the transgenic plant, and/or wherein the recombinant nucleic acid sequence is stably integrated into the chloroplast DNA of the transgenic plant. In some embodiments, the transgenic plant comprises at least about 10 copies, at least about 100 copies, at least about 1,000 copies, at least about 5,000 copies, at least about 10,000 copies, at least about 20,000 copies, at least about 30,000 copies, at least about 40,000 copies, or at least about 50,000 copies of the one or more recombinant nucleic acid sequences.

In certain embodiments, the transgenic plant is a grass (for example, a barely, a corn, a maize, an oat, a silver grass, a sugarcane, a rice, a rye, or a wheat), a legume (for example, an alfalfa, a bean, a chickpea, a clover, a lentil, a pea, or a peanut), a nightshade (for example, an eggplant, a pepper, a potato, a tobacco, or a tomato), an aster (for example, a lettuce, a chamomile, an artichoke, an endive, a lavender, a cotton, a sunflower), or an alga, a moss, or a liverwort. In some embodiments, the transgenic plant is a legume, and the legume is a soybean (Glycine max), a pea (Pisum satiyum), or a lupine (Lupimis mutabilis). In some embodiments, the transgenic plant is an aster, and the aster is a lettuce plant (i.e., a Lactuca species). In certain embodiments, the transgenic plant is a nightshade, and the nightshade is a tobacco plant (i.e., a Nicotiana species). In some embodiments, the tobacco plant is a nicotine-free tobacco plant. In some embodiments, the tobacco plant is a wild-type tobacco plant. In some embodiments, the myoglobin protein comprises at least about 0.1%, at least about 1.0%, at least about 10%, at least about 20%, at least about 30%, at least about 40%, at least about 50%, at least about 60%, at least about 70%, at least about 80%, or at least about 90% of the total soluble protein from the transgenic plant.

### BRIEF DESCRIPTION OF THE DRAWINGS

The following detailed description of the embodiments of the present invention can be best understood when read in conjunction with the following drawings, where like structure is indicated with like reference numerals and in which:

FIG. 1 shows a schematic diagram of an overview of an exemplary method for making transgenic plants expressing myoglobin and isolating myoglobin from the transgenic plants.

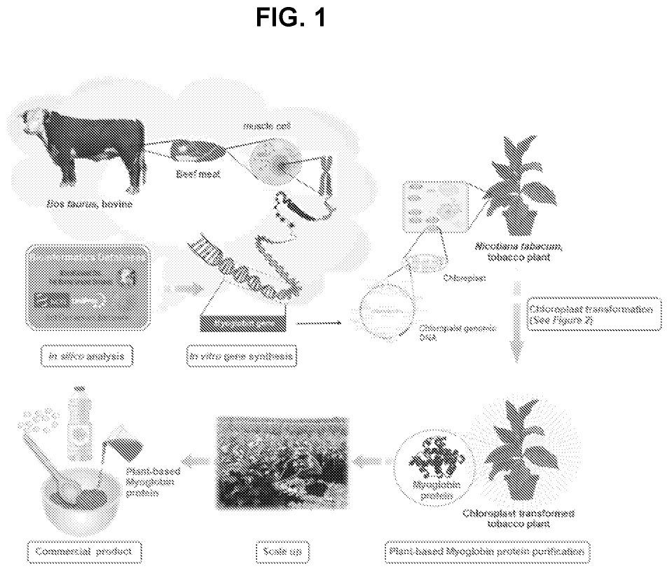

FIG. 2A-2C show a schematic diagram of an exemplary chloroplast genome transformation process.

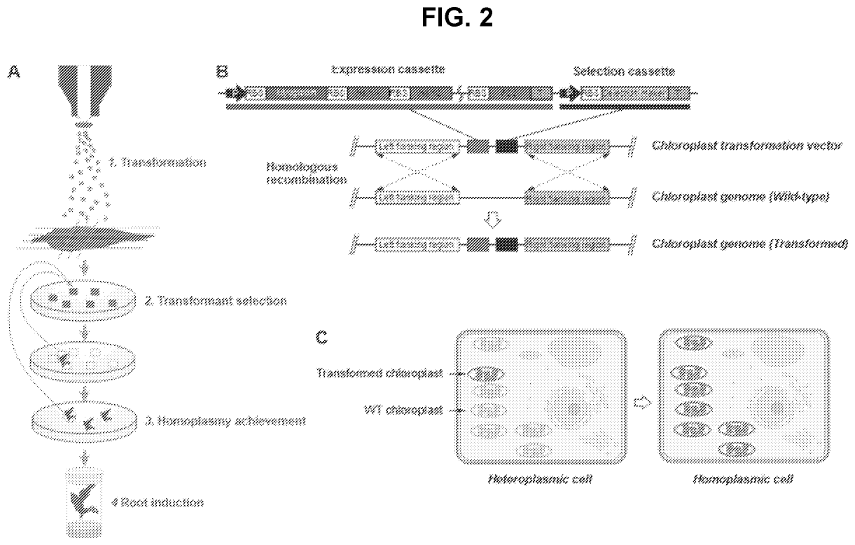

FIG. 3A-3C show a strategy for generation of transformants expressing Myoglobin using the HT72 recipient (psbH knock-out (KO) mutant). FIG. 3A) Physical map of the plastid transformation vector. The plastid(chloroplast) transformation vector contains the gene-of-interest (GOI) cassette which comprises a expression cassette having a coding sequence of the Bt Myoglobin, Ss Myoglobin and Tt Myoglobin (Myoglobin CDS) flanked by a chloroplast promoter/5′ untranslated region (5′UTR) element from a chloroplast gene (psaA Pro+S′UTR) and a transcription terminator from a chloroplast gene (rbel. 3′UTR), and homologous arms (i.e. Left flanking region and Right flanking region) which has a plastome sequence that includes psbH as a selectable marker. Introduction into the chloroplast of strain HT72 (a knockout strain in which psbh and the downstream region have been replaced with the aadA gene expression cassette (indicated as the insertion cassette for psbH KO) results in restoration of psbHl, introduction of the GOI and loss of aadA. As a result, transformants are capable of phototrophic growth on acetate-free medium (Hsm) and are sensitive to spectinomycin and streptomycin. FIG. 3B) Targeted insertion region of recipient (HT72) plastome. Red arrows indicate the four-primer set used to determine homoplasmy. FIG. 3C) Transformed plastome having Myoglobin expression cassette and a selection marker psbH.

FIG. 4 shows selection of transplastomic Chlamydomonas lines by photoautotrophic growth. Four isolated of Chlamydomonas reinhardtii transformants of Myoglobin gene (pKM017) line no 3, 5, 7, 9, two isolated vector control line no3 and 8, a recipient psbH KO mutant line (HT72) and Chlamydomonas reinhardfii wild-type strain (CC1690) were incubated on acetate-free medium (Hsm) under the phototrophic condition. CC1690 served as a positive control for autotrophy and HT72 as a negative control.

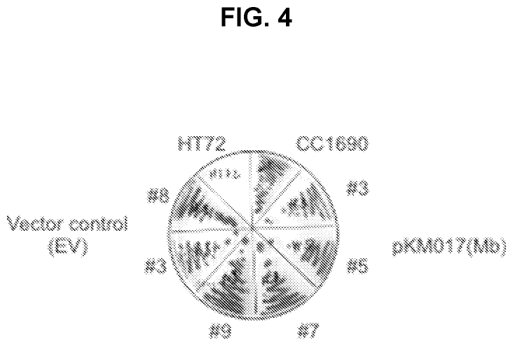

FIG. 5A-5C show PCR confirmation of Bt Myoglobin integration and homoplasmy of pKM017 transformants. Total genomic DNA from four isolated of Chlamydomonas reinhardtii transformants of Myoglobin gene (pKM017) lines, no 3, 5, 7 and 9, two isolated vector control lines, no. 3 and 8, a recipient pshH KO mutant line (HT72) and Chlamydomonas reinhardtii wild-type strain (CC1690) were isolated and subjected to PCR using the primer sets shown in FIGS. 3B and 3C. PCR products were visualized following electrophoresis in a 1% agarose gel containing ethidium bromide. FIG. 5A) PCR products were amplified using primer F1 and R1. FIG. 5B) PCR products were amplified using primer F1 and R2. FIG. 5C) PCR products were amplified using primer F1 and R3. FIG. 5D) PCR products were amplified using primer F1 and R4. The presence of a 2,583-bp band (FIG. 5A), 1,113 bp band (FIG. 5B) and 1,304-bp band (FIG. 5C) confirmed the successful integration of Bt Myoglobin, whereas the absence of the 878-bp band (FIG. 5D) that arises from the untransformed copies of the HT72 plastome indicates that the transformant lines are homoplasmic except for line3. EV: vector control line, Mb: pKM017 transformant line, CC1690: wild-type strain.

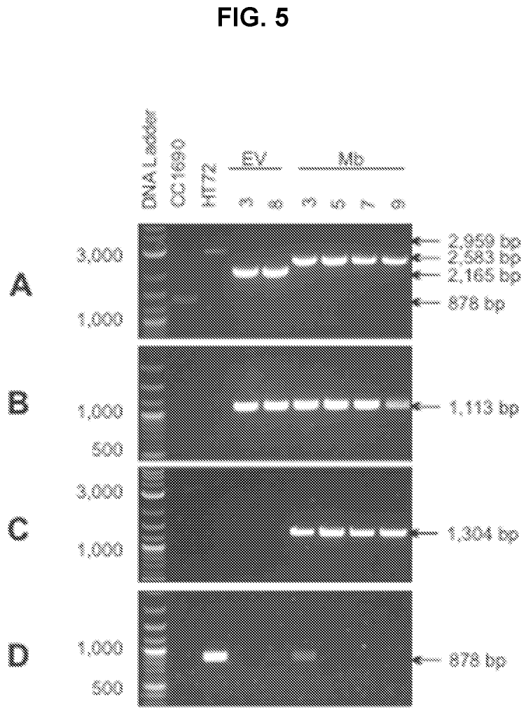

FIG. 6 shows Western blotting confirmation of Bt Myoglobin protein accumulation in pKM017 transformants. Total proteome were extracted from four isolated of Chlamydomonas reinhardtii transformants of Myoglobin gene (pKM017) lines no. 3, 5, 7 and 9, two isolated vector control lines, no. 3 and 8, a recipient psbh KO mutant line (HT72) and Chlamydomonas reinhardtii wild-type strain (CC1690) grown photoautotrophically in HSM medium at a light intensity of 60 μE m-2s-1 or mixotrophically in TAP medium at a light intensity of 60 μE m-2s-1. Proteins were separated on the SDS-PAGE gel and subjected to western blotting using the anti-Myoglobin antibody for detection of BI Myoglobin protein accumulation. The results demonstrate successful accumulation of Bt Myoglobin protein in all pKM017 (Mb) lines in both growth conditions and the accumulation levels of Bt Myoglobin proteins were significantly higher in those grown in the mixotrophic growth condition (TAP) than in those grown in the photoautotrophic condition (Hsm). The arrowhead indicates the Bt Myoglobin band and asterisks indicate nonspecific background.

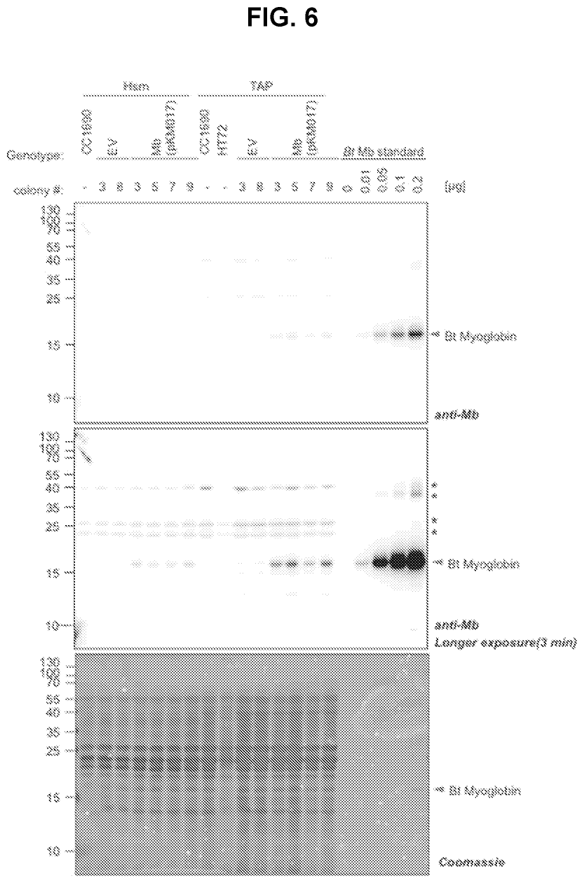

FIG. 7A-7C show a strategy for generation of marker-free transformants expressing Myoglobin in Nicotiana tabacum. FIG. 7A) Physical map of the plastid transformation vector. The plastid (chloroplast) transformation vector contains the gene-of-interest (GOI) cassette which comprises a expression cassette having a coding sequence of the Bt Myoglobin, Ss Myoglobin and Tt Myoglobin (Myoglobin CDS) flanked by a chloroplast promoter element from a chloroplast gene (ribosomal RNA operon) fused to the 5′ untranslated region (S′UTR) from gene 10 of phage T7 (Prrn+T7g10)) and a transcription terminator from a chloroplast gene (rhcL 3′UTR), and homologous arms (i.e. Left flanking region and Right flanking region) which are plastome sequences spanning between the trufM and irnG genes in the spacer region and the selection cassette having the selectable marker gene aadA driven by a chimeric ribosomal RNA operon promoter (Prrn) and fused to the 3′UTR from the plastid psbA gene and flanked with loxP sites to facilitate selectable marker gene removal by Cre-mediated site-specific recombination to create antibiotic resistant marker gene-free plants. FIG. 7B) Targeted insertion region of recipient plastome. Arrows indicate the primer set used to determine homoplasmy. FIG. 7C) Transformed plastome having Myoglobin expression cassette and a selection cassette. Red arrows indicate the four-primer set used to determine homoplasmy.

FIG. 8A-8B show generation of BI Myoglobin-expressing transplastomic Nicotiana tabacum. FIG. 8A) Primary transformants were selected on spectinomycin-containing (500 μg/mL) regeneration medium (RMOP). FIG. 8B) Double resistance tests on a medium containing spectinomycin (500 μg/mL) and streptomycin (500 μg/mL) were performed to eliminate lines with spontaneous mutations leading to antibiotic resistance. GFP control plants served as a positive control.

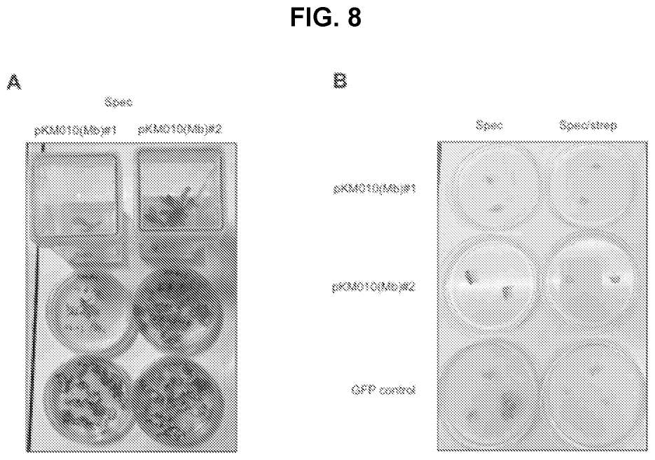

FIG. 9A-9B demonstrate Bt Myoglobin protein accumulation in pKM010 transformants. FIG. 9A) Two putative Nicotiana tabacum transformants of pKM010 and a GFP control line grown on MS medium with spectinomycin. FIG. 9B). Total proteomes were isolated from two putative Nicotiana tabacum transformants of Myoglobin gene (pKM010) lines, no. 1 and 2, an isolated GFP control line, no. 1 and a wild-type plant grown on MS medium with spectinomycin. Proteins were separated on the SDS-PAGE gel and subjected to western blotting using the anti-Myoglobin antibody for detection of Bt Myoglobin protein accumulation. The results indicate accumulation of Bf Myoglobin in pKM010 (Mb) line no. 2. The arrowhead indicates the Bt Myoglobin band.

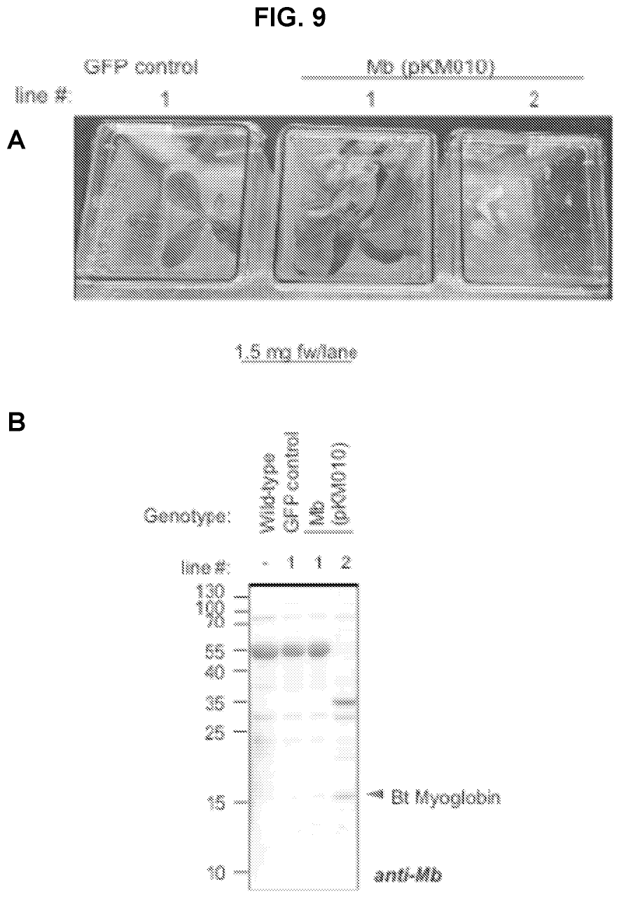

FIG. 10A-10C show a strategy for generation of marker-free transformants expressing Myoglobin in Lactuca sativa. FIG. 10A) Physical map of the plastid transformation vector. The plastid (chloroplast) transformation vector contains the gene-of-interest (GOI) cassette which comprises a expression cassette having a coding sequence of the Bi Myoglobin, S's Myoglobin and Tt Myoglobin (Myoglobin CDS) flanked by a chloroplast promoter element from a chloroplast gene (ribosomal RNA operon) fused to the 5′ untranslated region (S′UTR) from gene 10 of phage T7 (Prmn+17g10) and a transcription terminator from a chloroplast gene (rheL 3′UTR), and homologous arms (i.e. Left flanking region and Right flanking region) which are plastome sequences spanning between the trufM and trnG genes in the spacer region, and the selection cassette having the selectable marker gene aadA driven by a chimeric ribosomal RNA operon promoter (Prrn) and fused to the 3′UTR from the plastid psbA gene and flanked with loxP sites to facilitate selectable marker gene removal by Cre-mediated site-specific recombination and direct repeats consist of the upstream region of Nicotiana tabacum atpB gene for homologous recombination to loop out the selectable marker gene. FIG. 10B) Targeted insertion region of recipient plastome. Red arrows indicate the primer set used to determine homoplasmy. FIG. 10C) Transformed plastome having Myoglobin expression cassette and a selection cassette. Red arrows indicate the four-primer set used to determine homoplasmy.

Skilled artisans will appreciate that elements in the Figures are illustrated for simplicity and clarity and have not necessarily been drawn to scale. For example, the dimensions of some of the elements in the Figures can be exaggerated relative to other elements to help improve understanding of the embodiment(s) of the present invention.

### DETAILED DESCRIPTION

All publications, patents and patent applications cited herein are hereby expressly incorporated by reference for all purposes.

Methods well known to those skilled in the art can be used to construct genetic expression constructs and recombinant plants according to this invention. These methods include in vitro recombinant DNA techniques, synthetic techniques, in vivo recombination techniques, and PCR techniques. See, for example, techniques as described in Maniatis et al., 1989, MOLECULAR CLONING: A LABORATORY MANUAL, Cold Spring Harbor Laboratory, New York; Ausubel et al., 1989, CURRENT PROTOCOLS IN MOLECULAR BIOLOGY, Greene Publishing Associates and Wiley Interscience, New York, and PCR Protocols: A Guide to Methods and Applications (Innis et al., 1990, Academic Press, San Diego, CA); Liebers et al., “Regulatory Shifts in Plastid Transcription Play a Key Role in Morphological Conversions of Plastids during Plant Development”. Front. Plant Sci. (2017) 8:23.

Before describing the present invention in detail, a number of terms will be defined. As used herein, the singular forms “a”, “an”, and “the” include plural referents unless the context clearly dictates otherwise. For example, reference to a “nucleic acid” means one or more nucleic acids.

It is noted that terms like “preferably”, “commonly”, and “typically” are not utilized herein to limit the scope of the claimed invention or to imply that certain features are critical, essential, or even important to the structure or function of the claimed invention. Rather, these terms are merely intended to highlight alternative or additional features that can or cannot be utilized in a particular embodiment of the present invention.

For the purposes of describing and defining the present invention it is noted that the terms “increase”, “increases”, “increased”, “greater”, “higher”, and “lower” are utilized herein to represent non-quantitative comparisons, values, measurements, or other representations to a stated reference or control.

For the purposes of describing and defining the present invention it is noted that the term “substantially” is utilized herein to represent the inherent degree of uncertainty that can be attributed to any quantitative comparison, value, measurement, or other representation. The term “substantially” is also utilized herein to represent the degree by which a quantitative representation can vary from a stated reference without resulting in a change in the basic function of the subject matter at issue.

As used herein, the terms “polynucleotide”, “nucleotide”, “oligonucleotide”, and “nucleic acid” can be used interchangeably to refer to nucleic acid comprising DNA, RNA, derivatives thereof, or combinations thereof.

As used herein, the terms “polypeptide,” “protein,” “peptide,” and “amino acid sequence” are used interchangeably, and refer to a polymeric form of amino acids of any length, which can include coded and non-coded amino acids, chemically or biochemically modified or derivatized amino acids, and polypeptides having modified peptide backbones.

Unless otherwise apparent from the context, the term “about” encompasses insubstantial variations, such as values within a standard margin of error of measurement (e.g., SEM) of a stated value. The term “about” as used herein when referring to a measurable value such as a parameter, an amount, a temporal duration, is meant to encompass variations of +/−10% or less, +/−5% or less, or +/−1% or less or less of and from the specified value. It is to be understood that the value to which the modifier “about” refers is itself also disclosed.

### Production of Myoglobin in Transgenic Plants

Myoglobin can be produced in a transgenic plant. As used herein, the term “transgenic plant” is intended to refer to a plant or plant cell, the genome of which has been augmented by incorporation of one or more DNA sequences or one or more recombinant nucleic acid sequences. The term “transgene” as used herein refers to a DNA molecule artificially incorporated into the genome and/or plastome of a plant as a result of human intervention, such as by plant transformation methods. As used herein, the term “transgenic plant” refers to a plant comprising a transgene in its genome. As used herein, the term “transgenic plant” can also refer to a plant comprising a transgene in its chloroplast genome (i.e., chloroplast DNA or plastome). As a result of such genomic alteration, the transgenic plant is something distinctly different from the related wild-type plant and not naturally found in the wild-type plant. Transgenic plants of the invention comprise the one or more recombinant nucleic acid sequences provided by the invention. Such one or more recombinant nucleic acid sequences include, but are not limited to, genes that are not naturally present, DNA sequences that are not normally transcribed into RNA or translated into a protein (“expressed”), and other genes or DNA sequences that are desired to be introduced into the plant to produce the transgenic plant. It will be appreciated that the genome and/or plastome of a transgenic plant described herein is typically augmented through stable introduction of one or more recombinant genes. Generally, the introduced DNA is not originally resident in transgenic plant that is the recipient of the DNA, but it is within the scope of the invention to isolate a DNA segment from a given plant, and to subsequently introduce one or more additional copies of that DNA into the same plant, e.g., to enhance production of the product of a gene or alter the expression pattern of a gene. In some instances, the introduced one or more recombinant nucleic acid sequences can modify or replace an endogenous gene or DNA sequence by, e.g., homologous recombination or site-directed mutagenesis. In some embodiments, the transgenic plant is a legume, and the legume is a soybean (Glycine max), a pea (Pisum satiyum), or a lupine (Lupinus mutabilis). In some embodiments, the transgenic plant is an aster, and the aster is a lettuce plant (i.e., a Lactuca species). In certain embodiments, the transgenic plant is a nightshade, and the nightshade is a tobacco plant (i.e., a Nicotiana species). In some embodiments, the tobacco plant is a nicotine-free tobacco plant. In some embodiments, the tobacco plant is a wild-type tobacco plant.

The term “recombinant nucleic acid sequence” refers to a gene or DNA sequence that is introduced into a recipient plant, regardless of whether the same or a similar gene or DNA sequence may already be present in such a plant. “Introduced” or “augmented” in this context is known in the art to mean introduced or augmented by the hand of man. Thus, a recombinant nucleic acid sequence may be a DNA sequence from another species, or may be a DNA sequence that originated from or is present in the same species, but has been incorporated into a plant by recombinant methods to form a transgenic plant. It will be appreciated that a recombinant nucleic acid sequence that is introduced into a plant can be introduced to provide one or more copies of the DNA to thereby permit overexpression or modified expression of the gene product of that DNA. In some embodiments, the DNA is a cDNA copy of an mRNA transcript of a gene produced in a cell. In some embodiments, the DNA is codon optimized. As used herein, the terms “codon optimization” and “codon optimized” refer to a technique to maximize protein expression in a desired plant species by increasing the translation efficiency of a particular gene. Codon optimization can be achieved, for example, by transforming nucleotide sequences of one species into the genetic sequence of a different species. Optimal codons help to achieve faster translation rates and high accuracy. As a result of these factors, translational selection is expected to be stronger in highly expressed genes.

As used herein, “increased expression” or “overexpression” or “overexpressed” refer to increased expression of a gene or protein compared to normal, wild-type expression levels. In some embodiments, overexpression can be at least about 1.5-fold, at least about 2-fold, at least about 3-fold, at least about 4-fold, at least about 5-fold, at least about 10-fold, at least about 20-fold, at least about 30-fold, at least about 40-fold, or at least about 50-fold compared to a control level or amount. In certain embodiments, overexpression of a gene results in isolation of about 50 mg of the overexpressed protein per kilogram of fresh weight of tissue from the transgenic plant. In certain embodiments, overexpression of a gene results in isolation of about 100 mg of the overexpressed protein per kilogram of fresh weight of tissue from the transgenic plant. In certain embodiments, overexpression of a gene results in isolation of about 150 mg of the overexpressed protein per kilogram of fresh weight of tissue from the transgenic plant. In certain embodiments, overexpression of a gene results in isolation of about 200 mg of the overexpressed protein per kilogram of fresh weight of tissue from the transgenic plant. In certain embodiments, overexpression of a gene results in isolation of about 250 mg of the overexpressed protein per kilogram of fresh weight of tissue from the transgenic plant. In certain embodiments, overexpression of a gene results in isolation of about 300 mg of the overexpressed protein per kilogram of fresh weight of tissue from the transgenic plant. In certain embodiments, overexpression of a gene results in isolation of about 350 mg of the overexpressed protein per kilogram of fresh weight of tissue from the transgenic plant. In certain embodiments, overexpression of a gene results in isolation of about 400 mg of the overexpressed protein per kilogram of fresh weight of tissue from the transgenic plant. In certain embodiments, overexpression of a gene results in isolation of about 450 mg of the overexpressed protein per kilogram of fresh weight of tissue from the transgenic plant. In certain embodiments, overexpression of a gene results in isolation of about 500 mg of the overexpressed protein per kilogram of fresh weight of tissue from the transgenic plant. In certain embodiments, overexpression of a gene results in isolation of about 550 mg of the overexpressed protein per kilogram of fresh weight of tissue from the transgenic plant. In certain embodiments, overexpression of a gene results in isolation of about 600 mg of the overexpressed protein per kilogram of fresh weight of tissue from the transgenic plant. In certain embodiments, overexpression of a gene results in isolation of about 650 mg of the overexpressed protein per kilogram of fresh weight of tissue from the transgenic plant. In certain embodiments, overexpression of a gene results in isolation of about 700 mg of the overexpressed protein per kilogram of fresh weight of tissue from the transgenic plant. In certain embodiments, overexpression of a gene results in isolation of about 750 mg of the overexpressed protein per kilogram of fresh weight of tissue from the transgenic plant. In certain embodiments, overexpression of a gene results in isolation of about 800 mg of the overexpressed protein per kilogram of fresh weight of tissue from the transgenic plant. In certain embodiments, overexpression of a gene results in isolation of about 850 mg of the overexpressed protein per kilogram of fresh weight of tissue from the transgenic plant. In certain embodiments, overexpression of a gene results in isolation of about 900 mg of the overexpressed protein per kilogram of fresh weight of tissue from the transgenic plant. In some embodiments, the plant tissue is leaf tissue. In some embodiments, the plant tissue is seed. In some embodiments, the plant tissue is any part of the plant or the entire plant. In some embodiments, wherein the overexpressed protein comprises at least about 0.1%, at least about 1.0%, at least about 10%, at least about 20%, at least about 30%, at least about 40%, at least about 50%, at least about 60%, at least about 70%, at least about 80%, or at least about 90% of the total soluble protein from the transgenic plant. For the purposes of this disclosure, the original, normal, wild-type expression level might also be zero, i.e., absence of expression or immeasurable expression.

Reduction or elimination of gene expression may also comprise gene knock-out or knock-down. A “gene knock-out” refers to a plant cell or plant in which the expression of one or more genes is eliminated. For example, one or more genes involved in nicotine production in a tobacco plant can be knocked-out to eliminate nicotine production in the tobacco plant. In some embodiments, the transgenic plant can comprise a knock-out of one or more genes encoding magnesium chelatase enzymes. A “gene knock-down” refers to a plant cell or plant in which the level of one or more genes is reduced, but not completely eliminated. For example, one or more genes involved in nicotine production in a tobacco plant can be knocked-down to reduce nicotine production in the tobacco plant. In some embodiments, the transgenic plant comprises a knock-down of one or more genes encoding magnesium chelatase enzymes. In certain embodiments, the transgenic plants as described herein comprise modified, mutated, and/or knockouts or knockdowns of one or more genes encoding magnesium chelatase enzymes selected from the genes of Table 3 and/or Table 4.

The terms “plant promoter” or “promoter suitable for expression in plants” as used herein refers to a nucleic acid sequence comprising regulatory elements, which mediate the expression of a coding sequence in plant cells. For expression in plants, the nucleic acid molecule must be linked operably to or comprise a suitable promoter that expresses the gene at the right point in time and with the required spatial expression pattern. Promoters suitable for expression in plants comprise nucleic acid sequences that are able to direct the expression of a transgene in a plant. Examples of promoters suitable for expression in plants that are constitutive promoters that are transcriptionally active during most, but not necessarily all, phases of growth and development and under most environmental conditions, in at least one cell, tissue or organ, other promoters are inducible promoters, other examples are tissue specific promoters, still other examples are abiotic stress inducible promoters. In certain embodiments, the promoter can be a constitutive promoter such as the cauliflower mosaic virus (CaMV) 35S promoter, the mannopine synthase (MAS) promoter, the1′ or 2′ promoters derived from T-DNA of Agrobacterium tumefaciens, the figwort mosaic virus 34S promoter, actin promoters such as the rice actin promoter, or a ubiquitin promoter such as the maize ubiquitin-1 promoter. In certain embodiments, a plant specific constitutive promoter is active in chloroplasts of a plant. For example, plant specific constitutive promoter active in chloroplasts, can include, but are not limited to, N. tabacum rmn promoter, N. tabacum psbA promoter, N. tabacum rbcl, promoter, L. sativa rrn promoter, L. sativa psbA promoter and/or L. sativa rbel, promoter. The term “inducible promoter” refers to promoters that allow regulating gene expression levels at particular stages of plant development and in particular tissues of interest. Examples of inducible systems include AlcR/AlcA (ethanol inducible); GR fusions, GVG, and pOp/LbGR (dexamethasone inducible); XVE/OlexA (beta-estradiol inducible); and heat shock/cold induction. For expression in plants, the nucleic acid molecule can be operably linked to or comprise suitable untranslated regions such as S′UTR that regulates chloroplast mRNA translation and 3′UTR that control mRNA stability. Plant UTRs comprise nucleic acid sequences that are able to direct the expression of a transgene in a plant. Examples of plant UTRs can include, but are not limited to, N. tabacum psbA S′UTR, N. tabacum rbel. S′UTR, N. tabacum atpB S′UTR. L. sativa psbA S′UTR, L. sativa rbel S′UTR, L. sativa atpB S′UTR, the bacteriophage T7 gene 10 (T7g10) 5′ UTR, the Shine-Dalgarno (GGAGG) sequence, N. tabacum psbA 3′UTR, N. tabacum rps16 3′UTR, N. tabacum rhel. 3′UTR and N. tabacum petl) 3′UTR, L. sativa psbA 3′UTR, L. sativa rps 16 3′UTR, L. sativa rbel. 3′UTR and L. sativa petD) 3′UTR.

In some embodiments, the myoglobin gene is a bovine myoglobin gene (for example, bison, buffalo, cow, goat, sheep, or yak), an avian myoglobin gene (for example, chicken, duck, goose, guinea fowl, quail, pigeon, or turkey), a suine myoglobin gene (for example, boar or pig), or a fish myoglobin (for example, tuna, salmon, or eel). In some embodiments, the myoglobin gene is a myoglobin gene selected from Table I and/or wherein the myoglobin gene encodes a myoglobin protein selected from SEQ ID NO's 1-35. In certain embodiments, the myoglobin gene is a gene having at least 60% sequence identity, at least 65% sequence identity, at least 70% sequence identity, at least 75% sequence identity, at least 80% sequence identity, at least 85% sequence identity, at least 90% sequence identity, at least 95% sequence identity to the myoglobin gene from Bos taurus. In some embodiments, the myoglobin gene encodes a myoglobin protein having at least 60% sequence identity, at least 65% sequence identity, at least 70% sequence identity, at least 75% sequence identity, at least 80% sequence identity, at least 85% sequence identity, at least 90% sequence identity, at least 95% sequence identity to the myoglobin protein from Bos taurus (SEQ ID NO:04). In certain embodiments, the myoglobin gene is a gene having at least 60% sequence identity, at least 65% sequence identity, at least 70% sequence identity, at least 75% sequence identity, at least 80% sequence identity, at least 85% sequence identity, at least 90% sequence identity, at least 95% sequence identity to the myoglobin gene from Sus scrofa. In some embodiments, the myoglobin gene encodes a myoglobin protein having at least 60% sequence identity, at least 65% sequence identity, at least 70% sequence identity, at least 75% sequence identity, at least 80% sequence identity, at least 85% sequence identity, at least 90% sequence identity, at least 95% sequence identity to the myoglobin protein from Sus scrofa (SEQ ID NO:15). In certain embodiments, the myoglobin gene is a gene having at least 60% sequence identity, at least 65% sequence identity, at least 70% sequence identity, at least 75% sequence identity, at least 80% sequence identity, at least 85% sequence identity, at least 90% sequence identity, at least 95% sequence identity to the myoglobin gene from Thunnus thynnus. In some embodiments, the myoglobin gene encodes a myoglobin protein having at least 60% sequence identity, at least 65% sequence identity, at least 70% sequence identity, at least 75% sequence identity, at least 80% sequence identity, at least 85% sequence identity, at least 90% sequence identity, at least 95% sequence identity to the myoglobin protein from Thunnus thyunus (SEQ ID NO:35). In some embodiments, the myoglobin gene encodes a myoglobin protein having at least 60% sequence identity, at least 65% sequence identity, at least 70% sequence identity, at least 75% sequence identity, at least 80% sequence identity, at least 85% sequence identity, at least 90% sequence identity, or at least 95% sequence identity to a myoglobin protein encoded by a gene selected from the genes recited in Table 1.

TABLE 1
Exemplary myoglobin gene sequences.

| | | Similarity to | |
|---|---|---|---|
| Accession No. | Gene ID | P02192 | Species |
| A0A6P5BUC2 | 109559497 | Ident.: 100.0% | Bos indicus (Zebu) |
| L8IHL0 | 102282089 | Ident.: 100.0% | Bos matus (wild yak) |
| A0A4W2ICA1 | 113892672 | Ident.: 100.0% | Bos indicus × Bos taurus |
| | | | (Hybrid cattle) |
| P02192 | 280695 | Ident.: 100.0% | Bos taurus (Bovine) |
| Q2MJN4 | DQ324649.1 | Ident.: 100.0% | Bos mutus grunniens (Wild yak) |
| | | | (Bos grunniens) |
| P86873 | | Ident.: 100.0% | Bison bison (American bison) |
| | | | (Bos bison) |
| P02190 | 780509 | Ident.: 98.7% | Ovis aries (Sheep) |
| C0HJR0 | | Ident.: 98.7% | Rangifer tarandus (Reindeer) |
| | | | (Cervus tarandus) |
| P84997 | 102413227 | Ident.: 98.1% | Bubalus bubalis (Domestic water buffalo) |
| P02191 | | Ident.: 98.1% | Cervus elaphus (Red deer) |
| B7U9B5 | 100860833 | Ident.: 97.4% | Capra hircus (Goat) |
| P02181 | | Ident.: 88.3% | Inia geoffrensis (Amazon river dolphin) |
| P68082 | 100054434 | Ident.: 88.3% | Equus caballus (Horse) |
| P02170 | | Ident.: 88.3% | Oryctolagus cuniculus (Rabbit) |
| P02189 | 397467 | Ident.: 88.3% | Sus scrofa (Pig) |
| P02179 | AB271148.1 | Ident.: 85.1% | Balaenoptera acutorostrata (Common minke whale) |
| | | | (Balaena rostrata) |
| P02144 | 4151 | Ident.: 84.4% | Homo sapiens (Human) - for therapeutic |
| | | | or research purposes |
| P02194 | | Ident.: 80.5% | Macropus rufus (Red kangaroo) |
| | | | (Megaleia rufa) |
| A0A2P4TG07 | PPHD01000609.1 | Ident.: 73.4% | Bambusicola thoracicus (Chinese bamboo-partridge) |
| | | | (Perdix thoracica) |
| A0A7K9Y899 | VXAB01003205.1 | Ident.: 72.7% | Odontophorus gujanensis (marbled wood quail) |
| G1NJB6 | 100539180 | Ident.: 72.7% | Meleagris gallopavo (Wild turkey) |
| P02197 | 418056 | Ident.: 72.7% | Gallus gallus (Chicken) |
| A0A7K9V117 | VXAA01003071.1 | Ident.: 72.7% | Anseranas semipalmata (Magpie goose) |
| | | | (Anas semipalmata) |
| R0K422 | 101804689 | Ident.: 72.1% | Anas platyrhynchos (Mallard) |
| | | | (Anas boschas) |
| Q7LZM2 | | Ident.: 72.1% | Anas poccilorhyncha (Indian spot-billed duck) |
| P85077 | | Ident.: 70.1% | Struthio camelus (Common ostrich) |
| P02205 | AF291838 | Ident.: 42.6% | Thunnus albacares (Yellowfin tuna) |
| | | | (Neothunnus macropterus) |
| P68189 | AF291831 | | Thunnus thynnus (Atlantic bluefin tuna) |
| A0A1WSPRH3 | KT934795 | Ident.: 42.1% | Schizothorax labiatus (Kunar snowtrout) |
| Q617B0 | AB154423 | Ident.: 41.2% | Auxis rochei (Bullet tuna) |
| Q9DGI8 | AF291837 | Ident.: 44.4% | Katsuwonus pelamis (Skipjack tuna) (Bonito) |
| A0A0E3XU31 | 115541111 | Ident.: 43.2% | Gadus morhua (Atlantic cod) |
| Q9DG19 | AF291835 | Ident.: 41.2% | Scomber japonicus (Chub mackerel) |
| A0A075W2G8 | 118219805 | Ident.: 39.5% | Anguilla anguilla (European freshwater eel) |
| | | | (Muraena anguilla) |
| B9ENY2 | 100195613 | | Salmo salar (Atlantic salmon) |

### Exemplary Myoglobin Protein Sequences

>tr|A0A6P5BUC2|A0A6P5BUC2_BOSIN Myoglobin
OS = Bos indicus OX = 9915 GN = MB
PE = 3 SV = 1
(SEQ ID NO: 01)
MGLSDGEWQLVLNAWGKVEADVAGHGQEVLIRLFTGHPETLEKFD
KFKHLKTEAEMKASEDLKKHGNTVLTALGGILKKKGHHEAEVKHL
AESHANKHKIPVKYLEFISDAIIHVLHAKHPSDFGADAQAAMSKA
LELFRNDMAAQYKVLGFHG
>tr|L8IHL0|L8IHL0_9CETA Myoglobin
OS = Bos mutus
OX = 72004 GN = M91_19333
PE = 3 SV = 1
(SEQ ID NO: 02)
MGLSDGEWQLVLNAWGKVEADVAGHGQEVLIRLFTGHPETLEKFD
KFKHLKTEAEMKASEDLKKHGNTVLTALGGILKKKGHHEAEVKHL
AESHANKHKIPVKYLEFISDAIIHVLHAKHPSDFGADAQAAMSKA
LELFRNDMAAQYKVLGFHG
>tr|A0A4W2ICA1|A0A4W2ICA1_BOBOX Myoglobin
OS = Bos indicus x Bos taurus
OX = 30522 GN = MB PE = 3 SV = 1
(SEQ ID NO: 03)
MGLSDGEWQLVLNAWGKVEADVAGHGQEVLIRLFTGHPETLEKED
KFKHLKTEAEMKASEDLKKHGNTVLTALGGILKKKGHHEAEVKHL
AESHANKHKIPVKYLEFISDAIIHVLHAKHPSDFGADAQAAMSKA
LELFRNDMAAQYKVLGFHG
>sp|P02192|MYG_BOVIN Myoglobin
OS = Bos taurus
OX = 9913 GN = MB PE = 1 SV = 3
(SEQ ID NO: 04)
MGLSDGEWQLVLNAWGKVEADVAGHGQEVLIRLFTGHPETLEKED
KFKHLKTEAEMKASEDLKKHGNTVLTALGGILKKKGHHEAEVKHL
AESHANKHKIPVKYLEFISDAIIHVLHAKHPSDFGADAQAAMSKA
LELFRNDMAAQYKVLGFHG
>sp|Q2MJN4|MYG_BOSMU Myoglobin
OS = Bos mutus grunniens
OX = 30521 GN = MB
PE = 2 SV = 3
(SEQ ID NO: 05)
MGLSDGEWQLVLNAWGKVEADVAGHGQEVLIRLFTGHPETLEKFD
KFKHLKTEAEMKASEDLKKHGNTVLTALGGILKKKGHHEAEVKHL
AESHANKHKIPVKYLEFISDAIIHVLHAKHPSDFGADAQAAMSKA
LELFRNDMAAQYKVLGFHG
>sp|P86873|MYG_BISBI Myoglobin
OS = Bison bison
OX = 9901 GN = MB PE = 1 SV = 1
(SEQ ID NO: 06)
MGLSDGEWQLVLNAWGKVEADVAGHGQEVLIRLFTGHPETLEKFD
KFKHLKTEAEMKASEDLKKHGNTVLTALGGILKKKGHHEAEVKHL
AESHANKHKIPVKYLEFISDAIIHVLHAKHPSDFGADAQAAMSKA
LELFRNDMAAQYKVLGFHG
>sp|P02190|MYG_SHEEP Myoglobin
OS = Ovis aries
OX = 9940 GN = MB PE = 1 SV = 2
(SEQ ID NO: 07)
MGLSDGEWQLVLNAWGKVEADVAGHGQEVLIRLFTGHPETLEKFD
KFKHLKTEAEMKASEDLKKHGNTVLTALGGILKKKGHHEAEVKHL
AESHANKHKIPVKYLEFISDAIIHVLHAKHPSDFGADAQGAMSKA
LELFRNDMAAQYKVLGFQG
>sp|COHJRO|MYG_RANTA Myoglobin
OS = Rangifer tarandus
OX = 9870 GN = MB
PE = 1 SV = 1
(SEQ ID NO : 08)
MGLSDGEWQLVLNAWGKVEADVAGHGQEVLIRLFTGHPETLEKFD
KFKHLKTEAEMKASEDLKKHGNTVLTALGGILKKKGHHEAEVKHL
AESHANKHKIPVKYLEFISDAIIHVLHAKHPSDFGADAQGAMSKA
LELFRNDMAAQYKVLGFQG
>sp|P84997|MYG_BUBBU Myoglobin
OS = Bubalus bubalis
OX = 89462 GN = MB PE = 1
SV = 2
(SEQ ID NO: 09)
MGLSDGEWQLVLNAWGKVETDVAGHGQEVLIRLFTGHPETLEKFD
KFKHLKTEAEMKASEDLKKHGNTVLTALGGILKKKGHHEAEVKHL
AESHANKHKIPVKYLEFISDAIIHVLHDKHPSDFGADAQAAMSKA
LELFRNEMAAQYKVLGFHG
>sp|P02191|MYG_CEREL Myoglobin
OS = Cervus elaphus
OX = 9860 GN = MB PE = 1
SV = 2
(SEQ ID NO: 10)
MGLSDGEWQLVLNAWGKVEADVAGHGQEVLIRLFTGHPETLEKED
KFKHLKTEAEMKASEDLKKHGNTVLTALGGILKKKGHHEAEVKHL
AESHANKHKIPVKYLEFISDAIIHVLHAKHPSNEGADAQGAMSKA
LELFRNDMAAQY
> = p|B7U9B5|MYG_APRI Myoglobin
OS = Capra hircus
OX = 9925 GN = MB PE = 1 SV = 3
(SEQ ID NO: 11)
MGLSDGEWTLVLNANGKVEADVAGHGQEVLIRLFTGHPETLEKED
KFKHLKTGAEMKASEDLKKHGNTVLTALGGILKKKGHHEAEVKHL
AESHANKHKIPVKYLEFISDAIIHVLHAKHPSDFGADAQGAMSKA
LELFRNDMAAQYKVLGFQG
>sp|P02181|MYG_INIGE Myoglobin
OS = Inia geoffrensis
OX = 9725 GN = MB PE = 1
SV = 2
(SEQ ID NO: 12)
MGLSDGEWQLVLNIWGKVEADLAGHGQDVLIRLFKGHPETLEKFD
KFKHLKTEAEMKASEDLKKHGNTVITALGGILKKKGHHEAELKPL
AQSHATKHKIPIKYLEFISEAIIHVLHSRHPGDFGADAQAAMNKA
LELFRKDIAAKYKELGFHG
>sp|P68082|MYG_HORSE Myoglobin
OS = Equus caballus
OX = 9796 GN = MB PE = 1
SV = 2
(SEQ ID NO: 13)
MGLSDGEWQQVLNVWGKVEADIAGHGQEVLIRLFTGHPETLEKED
KFKALKTEAEMKASEDLKKAGTVVLTALGGILKKKGHHEAELKPL
AQSHATKHKIPIKYLEFISDAIIHVLHSKHPGDFGADAQGAMTKA
LELFRNDIAAKYKELGFQG
>sp|P02170|MYG_RABIT Myoglobin
OS = Oryctolagus cuniculus
OX = 9986 GN = MB
PR = 1 SV = 2
(SEQ ID NO: 14)
MGLSDAEWQLVLNVWGKVEADLAGHGQEVLIRLFHTHPETLEKED
KFKHLKSEDEMKASEDLKKHGNTVLTALGAILKKKGHHEAEIKPL
AQSHATKHKIPVKYLEFISEAIIHVLHSKHPGDFGADAQAAMSKA
LELFRNDIAAQYKELGFQG
>sp|P02189|MYG_PIG Myoglobin
OS = Sus scrofa
OX = 9823 GN = MB PE = 1 SV = 2
(SEQ ID NO: 15)
MGLSDGEWQLVLNVWGKVEADVAGHGQEVLIRLFKGHPETLEKFD
KEKHLKSEDEMKASEDLKKHGNTVLTALGGILKKKGHHEAELTPL
AQSHATKHKIPVKYLEFISEAIIQVLQSKHPGDFGADAQGAMSKA
LELFRNDMAAKYKELGEQG
>sp|202179|MYG_BALAC Myoglobin
OS = Balaenoptera acutorostrata
OX = 9767
GN = MB PE = 1 SV = 2
(SEQ ID NO: 16)
MVLSDAEWHLVLNIWAKVEADVAGHGQDILIRLFKGHPETLEKED
KFKHLKTEAEMKASEDLKKHGNTVLTALGGILKKKGHHEAELKPL
AQSHATKHKIPIKYLEFISDAIIHVLHSRHPAEFGADAQAAMNKA
LELFRKDIAAKYKELGFQG
>sp|P02144|MYG_HUMAN Myoglobin
OS = Homo sapiens
OX = 9606 GN = MB PE = 1 SV = 2
(SEQ ID NO: 17)
MGLSDGEWQLVLNVWGKVEADIPGHGQEVLIRLFKGHPETLEKFD
KFKHLKSEDEMKASEDLKKHGATVLTALGGILKKKGHHEAEIKPL
AQSHATKHKIPVKYLEFISECIIQVLQSKHPGDFGADAQGAMNKA
LELFREDMASNYKELGFQG
>spP02194|MYG_MACRU Myoglobin
OS = Macropus rufus
OX = 9321 GN = MB PE = 1
SV = 2
(SEQ ID NO: 18)
MGLSDGEWQLVLNIWGKVETDEGGHGKDVLIRLFKGHPETLEKFD
KFKHLKSEDEMKASEDLKKHGITVLTALGNILKKKGHHEAELKPL
AQSHATKHKIPVQFLEFISDAIIQVIQSKHAGNFGADAQAAMKKA
LELFRHDMAAKYKEFGFQG
>tr|A0A2P4TG07|A0A2P4TG07_BAMTH Myoglobin
OS = Bambusicola thoracicus
OX = 9083 GN = CIB84 000957 PE = 3 SV = 1
(SEQ ID NO: 19)
MGLSDQEWQQVLAIWGKVEADIAGHGHEVLMRLFRDHPETLDRED
KFKGLKTPDQMKGSEDLKKHGATVLTQLGKILKQKGNHEAELKPL
AQTHATKHKIPVKYLEFISEVIIKVIAEKHAADFGADSQAAMKKA
LELFRNDMASKYKEFGFQG
>tr|A0A7K9Y899|A0A7K9Y899_9GAL LMYG protein
(Fragment)
OS = Odontophorus gujanensis
OX = 886794 GN = Mb PE = 4 SV = 1
(SEQ ID NO: 20)
MGLSDQEWQQVLSIWGKVEADIAGRGHEVLMRLFHDHPETLDRFE
KFKGLKTPDQMKGSEDLKKHGATVLTQLGKILKQKGNHESELKPL
AQTHATKHKIPVKYLEFISEVIIKVIAEKHAADFGADSQAAMKKA
LELFRNDMAAKYKEFGFQG
>sp|G1NJB6|MYG_MELGA Myoglobin
OS = Meleagris gallopavo
OX = 9103 GN = MB
PE = 1 SV = 1
(SEQ ID NO: 21)
MGLSDQEWQQVLTIWGKVEADIAGHGHEVLMRLFHDHPETLDRFD
KFKGLKTPDQMKGSEDLKKHGATVLTQLGKILKQKGNHESELKPL
AQTHATKHKIPVKYLEFISEVIIKVIAEKHAADFGADSQAAMKKA
LELFRNDMASKYKEFGFQG
>sp|P02197|MYG_CHICK Myoglobin
OS = Gallus gallus
OX = 9031 GN = MB PE = 1
SV = 4
(SEQ ID NO: 22)
MGLSDQEWQQVLTIWGKVEADIAGHGHEVLMRLFHDHPETLDRED
KFKGLKTPDQMKGSEDLKKHGATVLTQLGKILKQKGNHESELKPL
AQTHATKHKIPVKYLEFISEVIIKVIAEKHAADFGADSQAAMKKA
LELFRNDMASKYKEFGFQG
>Er|A0A7K9V117|A0A7K9V117_ANSSE MYG protein
(Fragment)
OS = Anseranas semipalmata
OX = 8851 GN = Mb PE = 4 SV = 1
(SEQ ID NO: 23)
MGLSDQEWQHVLTIWGKVEADLAGHGHAVLIRLFQDHPETLDRFE
KFKGLKTPDQMKGSEDLKKHGVTVLTQLGKILKQKGNHEAELKPL
AQTHATKHKIPVKYLEFISEVIIKVIAEKHSADFGADSQAAMKKA
LELFRNDMASKYK
>tr|ROK422|ROK422_ANAPL Myoglobin (Fragment)
OS = Anas platyrhynchos
OX = 8839 GN = Anapl_09645 PB = 3 SV = 1
(SEQ ID NO: 24)
MGLSDQEWQQVLTIWGKVEADLAGHGHAVLMRLFQDHPETLDRFE
KFKGLKTPDQMKGSEDLKKHGVTVLTQLGKILKQKGNHEAELKPL
AQTHATKHKIPVKYLEFISEVIIKVIAEKHSADFGADSQAAMKKA
LELFRNDMASKYKEFGFQG
>sp|Q7LZM2|MYG_ANAPO Myoglobin
OS = Anas poecilorhyncha
OX = 75854 GN = MB
PE = 1 SV = 2
(SEQ ID NO: 25)
MGLSDQEWQQVLTIWGKVEADLAGHGHAVLMRLFQDHPETLDRFE
KFKGLKTPDQMKGSEDLKKHGVTVLTQLGKILKQKGNHEAELKPL
AQTHATKHKIPVKYLEFISEVIIKVIAEKHSADFGADSQAAMKKA
LELFRNDMASI
>sp|P85077|STRCA_Myoglobin
OS = Struthio camelus
OX = 8801 GN = MB PE = 1
SV = 1
(SEQ ID NO: 26)
MGLSDQEWQQVLTIWGKVESDIAGHGHAILMRLFQDHPETLDRFE
KFKGLTTPEQMKASEELKKHGVTVLTQLGKILKQKGKHEAELKPL
AQTHATKHKIPVKYLEFISEVIIKVIAEKHSADFGADSQAAMKKA
LELFRNDMASKYKEFGFQG
>sp|P02205|MYG_THUAL Myoglobin
OS = Thunnus albacares
OX = 8236 GN = mb
PE = 1 SV = 2
(SEQ ID NO: 27)
MADFDAVLKCWGPVEADYTTMGGLVLTRLFKEHPETQKLFPKFAG
IAQADIAGNAAISAHGATVLKKLGELLKAKGSHAAILKPLANSHA
TKHKIPINNFKLISEVLVKVMHEKAGLDAGGQTALRNVMGIIIAD
LEANYKELGFSG
>tr|A0A1W5PRH3|A0A1W5PRH3_9TELE Myoglobin
OS = Schizothorax labiatus
OX = 327704 GN = Mb PE = 2 SV = 1
(SEQ ID NO: 28)
MADHDLVLKCWGAIEADFTGHGGDVLNRLFKEHPETLKLFPKFVS
IPPSDLVGNAAVAAHGATVLTKLGELLKARGDHAALLKPLATTHA
NKHKIALNNFKLITEVLVKVMAEKAGLDAAGQSAFKRVMEAVIGD
IDTYYKEIGFAG
>sp|Q617BO|MYG_AUXRO Myoglobin
OS = Auxis rochei
OX = 217026 GN = mb PE = 2
SV = 3
(SEQ ID NO: 29)
MADFDAVLKCWGPVEADFNTVGGMVLARLFKDHPDTQKLFPKFAG
IAAGDLAGNAAVAAHGGTVLKKLGELLKAKGNHAAIIKPLANSHA
TKHKIPINNFKLITEALVHVMQEKAGLDAAGQTALRNVMGIVIAD
LEANYKELGFTG
>sp|Q9DGI8|MYG_KATPE Myoglobin
OS = Katsuwonus pelamis
OX = 8226 GN = mb
PE = 2 SV = 3
(SEQ ID NO: 30)
MADLDAVLKCWGAVEADFNTVGGLVLARLFKDHPETQKLFPKFAG
ITGDIAGNAAVAAHGATVLKKLGELLKAKGNHAAIIKPLANSHAK
QHKIPINNEKLITEALAHVLHEKAGLDAAGQTALRNVMGIVIADL
EANYKELGFTG
>tr|A0A0E3XU31|A0A0E3XU31_GADMO Myoglobin
(Fragment)
OS = Gadus morhua
OX = 8049 PE = 3 SV = 1
(SEQ ID NO: 31)
MADYDLVLRCWGPVEADYNTHGGLVLTRLFTEHPDTQKLFPKLAG
VGELAASVAVASHGATVLKKLGELLKTRGDHAALLKPLATSHANV
HKIPISNFKLITEVIAKHMAEKAGLDAAGQEALREVMSVVIADMD
ATYKELGFS
>sp|Q9DGI9|MYG_SCOJP Myoglobin
OS = Scomber japonicus
OX = 13676 GN = mb
PE = 2 SV = 3
(SEQ ID NO: 32)
MADFDAVLKFWGPVEADYDKIGNMVLTRLFTEHPDTQKLFPKFAG
IGLGDMAGNAAISAHGATVLKKLAEVLKAKGNHAGIIKPLANSHA
TKHKIAINNFKLITEIIVKVMQEKAGLDAGGQTALRNVMGVFIAD
MDANYKELGFSG
>tr|A0A075W2G8|A0A075W2G8_ANGAN Myoglobin
(Fragment)
OS = Anguilla anguilla
OX = 7936 PE = 2 SV = 1
(SEQ ID NO: 33)
FELVLKAWKPIEADLKGNGGVVLTRLFQEHPETQQLFPKFAAIAP
GDLAGNAAISEHGCTVLTKLGDLLHAKGNHADILKPLAKTHATQH
KIKLQNFQLITEVIVKLMGEKGVDAAGQEAVRKVMLAVIGDIDNF
YKVLGF
>tr|B9ENY2|B9ENY2_SALSA Myoglobin
OS = Salmo salar
OX = 8030 GN = MYG_PE = 2
SV = 1
(SEQ ID NO: 34)
MANYDMVLQCWEPVEADYNNHGGLVLSRLFAEHPETLTLFPKFAG
IAAGDLSGNAAVAAHGATVLRKLGELLNARGDHAATLKSLATTHA
NKHKIPLKNFTLITNIICKVMGEKAGLDEAGQEALRQVMGVIIAD
INVTYMELGFAG
>sp|P68189|MYG_THUTH Myoglobin
OS = Thunnus thynnus
OX = 8237 GN = mb PE = 1
SV = 2
(SEQ ID NO: 35)
MADFDAVLKCWGPVEADYTTIGGLVLTRLFKEHPETQKLFPKFAG
IAQADIAGNAAVSAHGATVLKKLGELLKAKGSHAAILKPLANSHA
TKHKIPINNFKLISEVLVKVMHEKAGLDAGGQTALRNVMGIIIAD
LEANYKELGESG

### Transgenic Plants

A number of plants are suitable for use in constructing the transgenic plants described herein. A plant species and strain selected for use in production of myoglobin can refer to live plants and live plant parts, including fresh fruit, vegetables and seeds. Also, the term “plant” as used herein encompasses whole plants, ancestors and progeny of the plants and plant parts, including seeds, shoots, stems, leaves, roots (including tubers), flowers, and tissues and organs, wherein each of the aforementioned comprise the one or more recombinant nucleic acid sequences of interest. The term “plant” can also encompasses plant cells, suspension cultures, callus tissue, embryos, meristematic regions, gametophytes, sporophytes, pollen and microspores, again wherein each of the aforementioned comprises the one or more recombinant nucleic acid sequences of interest. In some embodiments, the plants can include any organism with chloroplast DNA (ctDNA or cpDNA), a plastome, a chloroplast, an amyloplast, a chromoplast, an elaioplast, an etioplast, a gerontoplast, a leucoplast, and/or aproplastid.

Exemplary plant species are described in more detail below. However, it will be appreciated that other species can be suitable. In some embodiments, a suitable species of the transgenic plant is a grass. For example, Hordeum vulgare (barley), Zea mays (maize), Avena sativa (oat), Miscanthus sps. (silvergrass, for example Miscanthus sinensis and hybrids thereof, for example, Miscanthus/giganteus a hybrid of M. sinensis and M. sacchariflorus). Saccharum officinarum (sugarcane), a Oryza sativa (rice), a Zizania sps. (wild rice), Secale cereale (rye), a sorghum, Pennisetum glaucum (pearl millet) or a Triticum sps. (wheat, including wheat berries, and spelt). In some embodiments, a suitable species of the transgenic plant is a legume. For example, a Fabaceae (legume) selected from, Medicago sativa (alfalfa), Glycine max (soybean), a Phaseolus vulgaris (bean) varieties of common beans such as black beans, green beans, navy beans, northern beans, or pinto beans, Cicer arietinum (garbanzo or chick pea), Trifolium repens (clover), Vigna unguiculata (cow pea), Vigna radiata (Mung bean), Lupinus albus (lupin), Lupinus mutabilis, Lens culinaris (lentil), Pisum sativum (pea) varieties such as garden peas or sugar snap peas, or Arachis hypogaea (peanut)). In some embodiments, a suitable species of the transgenic plant is a nightshade. For example, a nightshade selected from, Solanum melongena (eggplant), Capsicum annuum (pepper), Solanum tuberosum (potato), Solanum lycopersicum (tomato), Petunia xhybrida (petunia), or Nicotiana tabacum (tobacco). In some embodiments, a suitable species of the transgenic plant is an Amaranthaceae (for example, Beta vulgaris (sugarbeet), Arabidopsis thaliana (Arabidopsis), an Asteraceae (for example, Lactuca sativa (lettuce), Artemisia annua (sweet wormwood), or Helianthus annuus (sunflower)), a Brassicaceae (for example, Brassica napus (Oilseed rape), Brassica oleracea (Cauliflower, Cabbage), Lesquerella fendleri (popweed)), a Chenopodium sp. (quinoa)), a Cucurbitaceae (for example, Momordica charantia (bitter melon)), a Grossypium spp. (cotton), Euglena gracilis, a Linaceae (for example, Limun usitatissimum (flax)), a Pedaliaceae (for example, Sesamum sp. (sesame)), Populus alba (poplar tree), or a Umbelliferae (for example, Daucus carota (carrot)). In some embodiments, an alga can be used. For example, a suitable species of the transgenic plant is a Bangiacede (for example, Pyropia yezoensis), a Chlamydomonas (for example, Chlamydomonas acidophila, Chlamydomonas caudate, or Chlamydomonas ehrenbergii, or Chlamydomonas elegans), a Cyanidiaceae (for example, Pyropia vezoensis), Cyanidioschizon merolae), a Dunaliellaceae (for example, Dunaliella tertiolecta), an Euglenaceae (for example, Euglena gracilis), a Haematococcaceae (for example, Haematococcus pluvialis), a Isochrysidaceae (for example, Tisochrysis luted), a Monodopsidaceae (for example, Nannochloropsis oceanica), a Phaeodactylaceae (for example, Phaeodactylum tricornunan), a Porphyridiophyceae (for example, Porphyridium sp. UTEX 637). In some embodiments, a moss can be used. For example, a Funariaceae (for example, Physcomitrella patens (moss). In some embodiments, a liverwort can be used. For example, a Marchantiaceae (for example, Marchantia polymorpha (umbrella liverwort)). It will be appreciated that any plant species could be used.

As used herein “grass” species refers to Poaceae or Graminede families of monocotyledonous flowering plants known as grasses, and can include cereal grasses, silvergrasses (Miscanthus sps.), bamboos and the grasses of natural grassland as well as species cultivated in lawns and pasture. Non-limiting examples of grass can include, for example, barely, corn, maize, oat, silvergrass, sugarcane, rice, rye, or wheat.

As used herein “legume” species refers to a plant in the family Fabaceae (or Leguminosae), or the fruit or seed of such a plant. Legumes are notable in that most of them have symbiotic nitrogen-fixing bacteria in structures called root nodules. Non-limiting examples of legume can include, for example, an alfalfa, a bean, a chickpea, a clover, a lentil, a lupine, a pea, a peanut, or a soybean.

As used herein “nightshade” species refers to a plant in the family Solanaceae, which are a family of flowering plants that ranges from annual and perennial herbs to vines, lianas, epiphytes, shrubs, and trees, and includes a number of agricultural crops, medicinal plants, spices, weeds, and ornamentals. Non-limiting examples of nightshade can include, for example, an eggplant, a pepper, a potato, a tobacco, or a tomato.

As used herein “aster” species refers to a plant in the family Asteraceae, which consists of over 32,000 known species of flowering plants in over 1,900 genera within the order Asterales. Commonly referred to as the aster, daisy, composite, or sunflower family. Most species of Asteraceae are annual, biennial, or perennial herbaceous plants, but there are also shrubs, vines, and trees. Asteraceae is an economically important family, providing food staples, garden plants, and herbal medicines. Non-limiting examples of aster can include, for example, a lettuce, a chamomile, an artichoke, an endive, a lavender, a cotton, or a sunflower.

The transgenic plants provided herein can be cultivated using conventional growing processes, including, inter alia, plant culture, plant tissue culture, field-grown, green house grown, or hydroponic cultivation. In some embodiments, the transgenic plants as disclosed herein may be used or cultivated in any manner.

The transgenic plants as disclosed herein comprise one more recombinant nucleic acid sequences expressing a myoglobin gene. In some embodiments, the one more recombinant nucleic acid sequences expressing a myoglobin gene can be introduced via viral vector-mediated transformation, electroporation, polyethylene glycol (PEG)-mediated transfection delivery method, nanoparticles (carbon nanotubes) delivery method or particle gun or biolistic delivery transformation (see for example, US 20170121724, U.S. Pat. Nos. 6,812,379, 7,767,885, 7,129,391, 7,135,620, 7,294,506, or US 20110072541; Lu et al., “Chloroplast transformation.” Methods Mol. Biol. 2006, 318, 285-303; O'neill et al., “Chloroplast transformation in plants: Polyethylene glycol (PEG) treatment of protoplasts is an alternative to biolistic delivery systems.” Plant J. 1993, 3, 729-738; and Kwak et al., “Chloroplast-selective gene delivery and expression in planta using chitosan-complexed single-walled carbon nanotube carriers.” Nat. Nanotechnol. 2019, 14, 447-455; incorporated by reference in their entirety). In certain embodiments, the one more recombinant nucleic acid sequence expressing a myoglobin gene is introduced via a biolistic delivery transformation system.

In certain embodiments, the one more recombinant nucleic acid sequences expressing a myoglobin gene is introduced in the chloroplast DNA (i.e., plastome of the plant). In certain embodiments, the one more recombinant nucleic acid sequences expressing a myoglobin gene is stably introduced in the chloroplast DNA (i.e., plastome of the plant). Chloroplasts are organelles that conduct photosynthesis in plant and algal cells. Chloroplasts have their own DNA, which can be abbreviated as ctDNA or cpDNA, and it is also known as the plastome. A chloroplast is also known as a plastid, characterized by its two membranes and a high concentration of chlorophyll. Other plastid types, such as the leucoplast and the chromoplast, contain little chlorophyll and do not carry out photosynthesis. In certain embodiments, the one more recombinant nucleic acid sequences expressing a myoglobin gene is introduced to any of the types of plastids (e.g. chloroplast, amyloplast, chromoplast, elaioplast, etioplast, gerontoplast, leucoplast, and/or proplastid).

In some embodiments, the one or more recombinant nucleic acid sequences disclosed herein are located within a genomic chromosome of the plant in addition to the one or more recombinant nucleic acid sequences stably integrated into the chloroplast DNA/plastid of a transgenic plant cell. For example, in certain embodiments, one or more recombinant nucleic acid sequences expressing a myoglobin gene are stably integrated into the chloroplast DNA/plastid, and one or more recombinant nucleic acid sequences expressing heme biosynthesis gene are transformed into the genomic DNA of the transgenic plant.

Methods for transformation of plants and/or plant cells are known in the art, and can include for example, any method by which DNA can be introduced into a cell (for example, where a recombinant DNA molecule is stably integrated into a plant chromosome). In certain embodiments, an Agrobacterium transformation system can be used for introducing one or more recombinant nucleic acid sequences into plants. Another exemplary method for introducing one or more recombinant nucleic acid sequences into plants is insertion of the one or more recombinant nucleic acid sequences into a plant genome at a pre-determined site by methods of site-directed integration. Site-directed integration may be accomplished by any method known in the art, for example, by use of zinc-finger nucleases, engineered or native meganucleases, TALE-endonucleases, or an RNA-guided endonuclease (for example a CRISPR/Cas9 system). Transgenic plants can be regenerated from a transformed plant cell by well-known methods of plant cell culture. A transgenic plant homozygous with respect to a transgene can be obtained by sexually mating (selfing) an independent segregant transgenic plant that contains a single exogenous gene sequence to itself, for example a RO or FO plant, to produce RI or FI seed. Plants grown from germinating RI or FI seed can be tested for heterozygosity.

To validate the homologous recombination of the one or more recombinant nucleic acid sequences into chloroplast genomes, transformed plant cells are grown on selective plates. Transformants can be selected and analyzed for integration and homoplasmicity after multiple rounds of growing single colonies under the selection marker resistance (for example, approximately four rounds). PCR, southern blot and sequencing can be used to confirm homoplasmic strains (i.e., that all copies of the chloroplast genome contained the target gene(s) from the one or more recombinant nucleic acid sequences).

As will be apparent to one skilled in the art, the particulars of the selection process for myoglobin expressing clones depend on the selectable markers. Selection promotes or permits proliferation of cells comprising the selectable marker while inhibiting or preventing proliferation of cells lacking the marker. For example, if a selectable marker is an antibiotic resistance gene, the transfected host cell population can be cultured in the presence of an antibiotic to which resistance is conferred by the selectable marker. In certain embodiments, the transgenic plants disclosed herein comprise one or more different selectable markers. For example, the transgenic plants can comprise two, three, four or five different selectable markers.

Generally after transformation, plant cells or cell groupings are selected for the presence of one or more selectable markers that are encoded by plant-expressible genes co-transferred with the one or more nucleic acids, following which, the transformed material can be regenerated into a whole plant. To select transgenic plants, the plant material obtained in the transformation is subjected to selective conditions so that transgenic plants can be distinguished from untransformed plants. A further possibility consists in growing the seeds, if appropriate after sterilization, on agar plates using a suitable selection agent so that only the transformed seeds can grow into plants. Transformants can be selected and analyzed for integration and homoplasmicity after multiple rounds of growing single colonies under the selection marker resistance (for example, approximately four rounds). PCR and sequencing can be used to confirm homoplasmic strains (i.e., that all copies of the chloroplast genome contained the target gene(s) from the one or more recombinant nucleic acid sequences). After selection, transgenic plant cells or transgenic plants can be cloned according to any appropriate method known in the art. In certain embodiments, the transgenic plant comprises at least about 10 copies, at least about 100 copies, at least about 1,000 copies, at least about 5,000 copies, at least about 10,000 copies, at least about 20,000 copies, at least about 30,000 copies, at least about 40,000 copies, or at least about 50,000 copies of the one or more recombinant nucleic acid sequences.

### Recombinant Nucleic Acid Sequences

Recombinant nucleic acid sequences or recombinant DNA constructs as disclosed herein are made by techniques known in the art and in various embodiments are included in plant transformation vectors, plasmids, or plastid DNA. Such recombinant nucleic acid sequences are useful for producing transgenic plants and/or transgenic cells and as such can also be contained in the genomic DNA of a transgenic plant, seed, cell, or plant part. In certain embodiments, the recombinant nucleic acid sequences or recombinant DNA constructs refer to chloroplast transformation vectors or plastid transformation vectors.

In some embodiments, the recombinant nucleic acid sequences disclosed herein are located within a chromosome (genomic) or plastid of a transgenic plant cell. Methods for constructing chloroplast transformation vectors or plastid transformation vectors are known in the art. Plant chloroplast transformation vectors or plastid transformation vectors typically include, but are not limited to: a suitable promoter for the expression of an operably linked DNA, an operably linked recombinant DNA construct, a ribosomal protein binding site (which may be included in S′UTR sequence) and a polyadenylation signal (which may be included in a 3′UTR sequence). Promoters useful in practicing the invention include those that function in a plant for expression of an operably linked gene. Such promoters are well known in the art and can include those that are inducible, viral, synthetic, constitutive, temporally regulated, spatially regulated, and/or spatio-temporally regulated. Additional optional components include, but are not limited to, one or more of the following targets: 5′ UTR, enhancer, cis-acting target, intron, signal sequence, transit peptide sequence, one or more genes encoding one or more enzymes in the heme biosynthesis pathway, one or more targeting sequences for homologous recombination in the transgenic plant chloroplast DNA, and one or more selectable marker genes. In some embodiments, the recombinant nucleic acid sequences further comprises a localization sequence that can be used to direct one or more target proteins to a particular intracellular compartment. For example, the recombinant nucleic acid sequences can comprise a localization sequence that directs the expressed protein to the endoplasmic reticulum (ER), mitochondria, plastids (such as chloroplasts), the vacuole, the Golgi apparatus, protein storage vesicles (PSV), extracellular domain (apoplast) and membranes.

In some embodiments, the one or more recombinant nucleic acid sequences disclosed herein are located within a genomic chromosome of the plant and the chloroplast DNA/plastid of a transgenic plant cell. For example, one or more recombinant nucleic acid sequences expressing the myoglobin gene are stably integrated into the chloroplast DNA/plastid, and one or more recombinant nucleic acid sequences expressing heme biosynthesis gene are transformed into the genomic DNA of the transgenic plant.

In some embodiments, the one or more recombinant nucleic acid sequences disclosed herein comprise a myoglobin gene encoding myoglobin protein. In some embodiments, the myoglobin gene is a bovine myoglobin gene (for example, bison, buffalo, cow, goat, sheep, or yak), an avian myoglobin gene (for example, chicken, duck, goose, guinea fowl, quail, pigeon, or turkey), a suine myoglobin gene (for example, boar or pig), or a fish myoglobin (for example, tuna, salmon, or eel). In some embodiments, the myoglobin gene is a myoglobin gene selected from Table I and/or the myoglobin gene encodes a myoglobin protein selected from SEQ ID NO's 1-35. In certain embodiments, the myoglobin gene is a gene having at least 60% sequence identity, at least 65% sequence identity, at least 70% sequence identity, at least 75% sequence identity, at least 80% sequence identity, at least 85% sequence identity, at least 90% sequence identity, at least 95% sequence identity to the myoglobin gene from Bos taurus. In some embodiments, the myoglobin gene encodes a myoglobin protein having at least 60% sequence identity, at least 65% sequence identity, at least 70% sequence identity, at least 75% sequence identity, at least 80% sequence identity, at least 85% sequence identity, at least 90% sequence identity, at least 95% sequence identity to the myoglobin protein from Bos taurus (SEQ ID NO:04). In certain embodiments, the myoglobin gene is a gene having at least 60% sequence identity, at least 65% sequence identity, at least 70% sequence identity, at least 75% sequence identity, at least 80% sequence identity, at least 85% sequence identity, at least 90% sequence identity, at least 95% sequence identity to the myoglobin gene from Sus scrofa. In some embodiments, the myoglobin gene encodes a myoglobin protein having at least 60% sequence identity, at least 65% sequence identity, at least 70% sequence identity, at least 75% sequence identity, at least 80% sequence identity, at least 85% sequence identity, at least 90% sequence identity, at least 95% sequence identity to the myoglobin protein from Sus scrofa (SEQ ID NO:15). In certain embodiments, the myoglobin gene is a gene having at least 60% sequence identity, at least 65% sequence identity, at least 70% sequence identity, at least 75% sequence identity, at least 80% sequence identity, at least 85% sequence identity, at least 90% sequence identity, at least 95% sequence identity to the myoglobin gene from Thunnus thynnus. In some embodiments, the myoglobin gene encodes a myoglobin protein having at least 60% sequence identity, at least 65% sequence identity, at least 70% sequence identity, at least 75% sequence identity, at least 80% sequence identity, at least 85% sequence identity, at least 90% sequence identity, at least 95% sequence identity to the myoglobin protein from Thunnus thynnus (SEQ ID NO:35). In some embodiments, the myoglobin gene encodes a myoglobin protein having at least 60% sequence identity, at least 65% sequence identity, at least 70% sequence identity, at least 75% sequence identity, at least 80% sequence identity, at least 85% sequence identity, at least 90% sequence identity, or at least 95% sequence identity to a myoglobin protein as encoded by a gene selected from the genes recited in Table I and/or one of the myoglobin protein sequences of SEQ ID NO:1-35.

In certain embodiments, the transgenic plants and recombinant nucleic acid sequences comprise genes for increasing the biosynthesis of heme for incorporation into heme-containing proteins. In certain embodiments, the genes for the heme biosynthesis pathway are overexpressed or included in one or more copies. Heme biosynthesis pathway proteins can be from a plant such as a grass. For example, Hordeum vulgare (barley), Zea mays (maize), Avena sativa (oat), Miscanthus sps. (silvergrass, for example Miscanthus sinensis and hybrids thereof, for example, Miscanthus x giganteus a hybrid of M. sinensis and M. sacchariflorus), Saccharum officinarum (sugarcane), a Oryza sativa (rice), a Zizania sps. (wild rice), Secale cereale (rye), a sorghum, Pennisetum glaucum (pearl millet) or a Triticum sps. (wheat, including wheat berries, and spelt). In some embodiments, a suitable species is a legume. For example, a Fabaceae (legume) selected from, Medicago sativa (alfalfa), Glycine max (soybean), a Phaseolus vulgaris (bean) varieties of common beans such as black beans, green beans, navy beans, northern beans, or pinto beans, Cicer arietiman (garbanzo or chick pea), Trifolium repens (clover), Vigna unguiculata (cow pea), Vigna radiata (Mung bean), Lupinus albus (lupin), Lens culinaris (lentil), Lupinus mutabilis, Pisum sativum (pea) varieties such as garden peas or sugar snap peas, or Arachis hypogaea (peanut)). In some embodiments, a suitable species is a nightshade. For example, a nightshade selected from, Solanum melongena (eggplant), Capsicum anmam (pepper), Solanum tuberosum (potato), Solanum lycopersicum (tomato), Petunia xhybrida (petunia), or Nicotiana tabacum (tobacco). In some embodiments, a suitable species is an Amaranthaceae (for example, Beta vulgaris (sugarbeet), Arabidopsis thaliana (Arabidopsis), an Asteraceae (for example, Lactuca sativa (lettuce), Artemisia annua (sweet worniwood), or Helianthus annuus (sunflower)), a Brassicaceae (for example, Brassica napus (Oilseed rape), Brassica oleracea (Cauliflower, Cabbage), Lesquerella fendleri (popweed)), a Chenopodium sp. (quinoa)), a Cucurbitaceae (for example, Momordica charantia (bitter melon)), a Gossypium spp. (cotton), Eugiena gracilis, a Linaceae (for example, Linum usitatissimum (flax)), a Pedaliaceae (for example, Sesamum sp. (sesame)), Populus alba (poplar tree), or a Umbelliferae (for example, Daucus carota (carrot)). In some embodiments, an alga can be used. For example, a suitable species is a Chlamydomonas (for example, Chlamydomonas acidophila, Chiamydomonas candate, or Chlamydomonas ehrenbergii, or Chlamydomonas elegans), and an Euglenaceae (for example, Euglena gracilis). In some embodiments, heme biosynthesis pathway proteins can be from an alga. For example, a Bangiaceae (for example, Pyropia yezoensis), a Chlamydomonas (for example, Chlamydomonas acidophila, Chlamydomonas caudate, or Chlamydomonas ehrenbergii, or Chlamydomonas elegans), a Cyanidiaceae (for example, Pyropia yezoensis), Cyanidioschizon merolae), a Dunaliellaceae (for example, Dunaliella tertiolecta), an Euglenaceae (for example, Euglena gracilis), a Haematococcaceae (for example, Haematococcus pluvialis), a Isochrysidaceae (for example, Tisochrysis lutea), a Monodopsidaceae (for example, Nannochloropsis oceanica), a Phaeodactylaceae (for example, Phaeodactylum tricornutum), a Porphyridiophyceae (for example, Porphyridium sp. UTEX 637). In some embodiments, heme biosynthesis pathway proteins can be from a moss. For example, a Funariaceae (for example, Physcomitrella patens (moss). In some embodiments, heme biosynthesis pathway proteins can be from a liverwort. For example, a Marchantiaceae (for example, Marchantia polymorpha (umbrella liverwort)).

In certain embodiments, the transgenic plants and recombinant nucleic acid sequences as described herein comprise genes encoding enzymes in the heme biosynthesis pathway from Nicotiana tabacum (for example, ferrochelatase-2, accession number A0AIS3YUH8, Gene ID LOC107779891). In certain embodiments, the one or more endogenous heme biosynthesis genes are orthologs of heme biosynthesis genes from Arabidopsis (e.g. ferrochelatase-1 or ferrochelatase-2 (FC1 (At5g26030, GenBank AED93514.1) or FC2 (At2g30390, GenBank AAB63095.1)) In certain embodiments, the one or more endogenous heme one or more endogenous heme biosynthesis genes are orthologs of heme biosynthesis genes from Lactuca (e.g. ferrochelatase Loc111894117 (see also Table 2).

TABLE 2
Exemplary heme biosynthesis sequences

| | | | | | | Nicotiana | Nicotiana |
|---|---|---|---|---|---|---|---|
| | | Gene Name | | PANTHER | | tabacum | tabacum |
| | Mapped | Gene Symbol | PANTHER | Protein | | ortholog | ortholog |
| Gene ID | IDs | Ortholog | Family/Subfamily | Class | Species | Uniprot ID | Gene ID |
| ARATH\|TAIR = | P42804 | Glutamyl-IRNA | GLUTAMYL-TRNA | — | Arabidopsis | AOA1S3XBH0 | LOC107763283 |
| locus = 2016605\| | | reductase 1, | REDUCTASE 1, | | thaliana | | |
| UniProtKB = | | chloroplastic | CHLOROPLASTIC | | | | |
| P42804 | | HEMA1 | (PTHR43120: SF1) | | | | |
| ARATH\|TAIR = | Q9LR75 | Copro- | OXYGEN- | oxidase | Arabidopsis | A0A1S3XMU0 | LOC107766888 |
| locus = 2825062\| | | porphyrinogen-III | DEPENDENT | | thaliana | | |
| UniProtKB = | | oxidase 1, | COPRO- | | | | |
| Q9LR75 | | chloroplastic | PORPHYRINOGEN- | | | | |
| | | CPX1 | III OXIDASE. | | | Q42946 | LOC107815524 |
| | | | MITOCHONDRIAL | | | A0A1S4C5X4 | LOC107815524 |
| | | | (PTHR10755: SF0) | | | | |
| ARATH\|TAIR = | Q93296 | Copro- | OXYGEN- | oxidase | Arabidopsis | A0A1S3XMU0 | LOC107766888 |
| locus = | | porphyrinogen- | DEPENDENT | | thaliana | | |
| 505006431\| | | III | COPRO- | | | | |
| UniProtKB = | | oxidase 2, | PORPHYRINOGEN- | | | | |
| Q93Z96 | | chloroplastic | III OXIDASE, | | | | |
| | | CPX2 | MITOCHONDRIAL | | | Q42946 | LOC107815524 |
| | | | (PTHR10755: SF0) | | | A0A1S4C5X4 | LOC107815524 |
| ARATH\|TAIR = | Q9SFH9 | Delta- | DELTA- | dehydratase | Arabidopsis | A0A1S4A383 | LOC107793287 |
| locus = 2205035\| | | aminolevulinic | AMINOLEVULINIC | | thaliana | | |
| UniProtKB = | | acid | ACID | | | | |
| Q9SFH9 | | dehydratase 1, | DEHYDRATASE | | | | |
| | | chloroplastic | (PTHR11458: SF0) | | | | |
| | | HEMB1 | | | | A0A1S4AKW2 | LOC107798564 |
| ARATH\| | O04921 | Ferrochelatase-2, | FERROCHELATASE, | lyase | Arabidopsis | A0A1S4DRN8 | LOC107832463 |
| EnsemblGenomes = | | chloroplastic | MITOCHONDRIAL | | thaliana | | |
| AT2G30390\| | | FC2 | (PTHR11108: SF1) | | | A0A1S3YUH8 | LOC107779891 |
| UniProtKB = | | | | | | | |
| O04921 | | | | | | | |
| ARATH\|TAIR = | Q9FMJ4 | Putative heme | RADICAL | e | Arabidopsis | A0A1S4C400 | LOC107814909 |
| locus = 2161897\| | | chaperone | S-ADENOSYL | | thaliana | | |
| UniProtKB = | | MDC12.26 | METHIONINE | | | A0A1S4CMH5 | LOC107820625 |
| Q9FMJ4 | | | DOMAIN- | | | | |
| | | | CONTAINING | | | | |
| | | | PROTEIN 1, | | | | |
| | | | MITOCHONDRIAL | | | | |
| | | | (PTHR13932: SF5) | | | | |
| ARATH\|TAIR = | Q9SJX1 | Probable glutamyl- | GLUTAMYL-TRNA | — | Arabidopsis | A0A1S3XBH0 | LOC107763283 |
| locus = 2042516\| | | tRNA reductase 3, | REDUCTASE 3, | | thaliana | | |
| UniProtKB = | | chloroplastic | CHLOROPLASTIC- | | | | |
| Q9SJX1 | | HEMA3 | RELATED | | | | |
| | | | (PTHR43120: SF6) | | | | |
| ARATH\|TAIR = | Q43316 | Porphobilinogen | PORPHO- | deaminase | Arabidopsis | A0A1S4AYZ7 | LOC107802823 |
| locus = 2150758\| | | deaminase, | BILINOGEN | | thaliana | | |
| UniProtKB = | | chloroplastic | DEAMINASE | | | | |
| Q43316 | | HEMC | (PTHR11557: SF0) | | | A0A1S4DII9 | LOC107830132 |
| | | | | | | A0A1S3XSC6 | LOC107767993 |
| | | | | | | A0A1S4AY65 | LOC107802603 |
| ARATH\|TAIR = | Q93ZB6 | Uroporphyrinogen | URO- | methyl- | Arabidopsis | A0A1S4AR72 | LOC107800512 |
| locus = 2086300\| | | decarboxylase 1, | PORPHYRINOGEN | transferase | thaliana | | |
| UniProtKB = | | chloroplastic | DECARBOXYLASE | | | | |
| Q93ZB6 | | HEME1 | 1; | | | A0A1S3XIN7 | LOC107765624 |
| | | | CHLOROPLASTIC | | | | |
| | | | (PTHR21091: SF167) | | | | |
| ARATH\|TAIR = | O48721 | Uroporphyrinogen- | URO- | — | Arabidopsis | A0A1S4DAY9 | LOC107827902 |
| locus = 2066256\| | | III synthase, | PORPHYRINOGEN- | | thaliana | | |
| UniProtKB = | | chloroplastic | II SYNTHASE, | | | | |
| O48721 | | UROS | CHLOROPLASTIC | | | A0A1S3XCK3 | LOC107763502 |
| | | | (PTHR38042: SF1) | | | | |
| ARATH\|TAIR = | O48782 | Heme oxygenase | HEME | oxygenase | Arabidopsis | A0A1S4A289 | LOC107792958 |
| locus = 2005513\| | | 1, chloroplastic | OXYGENASE 1, | | thaliana | | |
| UniProtKB = | | HO1 | CHLOROPLASTIC- | | | A0A1S4CGA1 | LOC107818591 |
| O48782 | | | RELATED | | | | |
| | | | (PTHR35703: SF2) | | | | |
| ARATH\|TAIR = | Q42522 | Glutamate-1- | GLUTAMATE-1- | matase | Arabidopsis | A0A1S3XXN7 | LOC107769957 |
| locus = 2114520\| | | semialdehyde 2,1- | SEMI- | | thaliana | | |
| UniProtKB = | | aminomutase 2, | ALDEHYDE 2, 1- | | | | |
| Q42522 | | chloroplastic | AMINOMUTASE 1, | | | | |
| | | GSA2 | CHLOROPLASTIC- | | | P31593 | LOC107769957 |
| | | | RELATED | | | A0A1S4D6G2 | LOC107826543 |
| | | | (PTHR43713: SF3) | | | | |
| ARATH\|TAIR = | O22886 | Uroporphyrinogen | URO- | methyl- | Arabidopsis | A0A1S4BE88 | LOC107807289 |
| focus = 2061903\| | | decarboxylase 2, | PORPHYRINOGEN | transferase | thaliana | | |
| UniProtKB = | | chloroplastic | DECARBOXYLASE | | | | |
| O22886 | | HEME2 | (PTHR21091: SF169) | | | A0A1S4C086 | LOC107813684 |
| | | | | | | Q42967 | LOC1027854239 |
| | | | | | | A0A1S4DF13 | LOC107829118 |
| | | | | | | A0A1S3XTT0 | LOC107768723 |
| ARATH\|TAIR = | P55826 | Protoporphyrinogen | PROTO- | oxidase | Arabidopsis | A0A1S3XTT0 | LOC107768723 |
| locus = 2133397\| | | oxidase l, | PORPHYRINOGEN | | thaliana | | |
| UniProtKB = | | chloroplastic | OXIDASE | | | | |
| P55826 | | PPOX1 | (PTHR42923: SF3) | | | Q9SBI4 | LOC107827378 |
| | | | | | | A0A1S3YXC4 | LOC107780878 |
| ARATH\|TAIR = | Q9SR43 | Phytochromobilin: | PHYTO- | oxido- | Arabidopsis | A0A1S3X5V2 | LOC107761635 |
| focus = 2083484\| | | ferredoxin | CHROMOBILIN: | reductase | thaliana | | |
| UniProtKB = | | oxidoreductase, | FERREDOXIN | | | | |
| Q9SR43 | | chloroplastic | OXIDO- | | | | |
| | | HY2 | REDUCTASE, | | | A0A1S4CWC5 | LOC107823317 |
| | | | CHLOROPLASTIC | | | | |
| | | | (PTHR34557: SF1) | | | | |
| ARATH\|TAIR = | P49294 | Glutamyl-tRNA | GLUTAMYL-TRNA | — | Arabidopsis | A0A1S3XBH0 | LOC107763283 |
| locus = 2024392\| | | reductase 2, | REDUCTASE 2, | | thaliana | | |
| UniProtKB = | | chloroplastic | CHLOROPLASTIC | | | | |
| P49294 | | HEMA2 | (PTHR43120: SF14) | | | | |
| ARATH\|TAIR = | P42799 | Glutamate-1- | GLUTAMATE-1- | mutase | Arabidopsis | A0A1S3XXN7 | LOC107769957 |
| locus = 2160554\| | | semialdehyde 2,1- | SEMI- | | thaliana | | |
| UniProtKB = | | aminomutase 1, | ALDEHYDE 2,1- | | | | |
| P42799 | | chloroplastic | AMINOMUTASE 1, | | | | |
| | | GSA1 | CHLOROPLASTIC- | | | P31593 | LOC107769957 |
| | | | RELATED | | | A0A1S4D6G2 | LOC107826543 |
| | | | (PTHR43713: SF3) | | | | |
| ARATH\|TAIR = | Q8S9J1 | Protoporphyrinogen | PROTO- | oxidase | Arabidopsis | A0A1S4C6L8 | LOC107815753 |
| locus = 2145603\| | | oxidase 2, | PORPHYRINOGEN | | thaliana | | |
| UniProtKB = | | chloroplastic/ | OXIDASE 2, | | | | |
| Q8S9J1 | | mitocbondrial | CHLOROPLASTIC/ | | | | |
| | | PPOX2 | MITOCHONDRIAL | | | A0A1S3X536 | LOC107761284 |
| | | | (PTHR42923: SF32) | | | O24164 | LOC3914403 |
| ARATH\|TAIR = | Q94LA4 | Probable delta- | DELTA- | dehydratase | Arabidopsis | A0A1S4A3B3 | LOC107793287 |
| locus = 2823624\| | | aminolevulinic | AMINOLEVULINIC | | thaliana | | |
| UniProtKB = | | acid dehydratase 2, | ACID | | | | |
| Q94LA4 | | chloroplastic | DEHYDRATASE 2, | | | | |
| | | HEMB2 | CHLOROPLASTIC- | | | A0A1S4AKW2 | LOC107798564 |
| | | | RELATED | | | | |
| | | | (PTHR11458: SF3) | | | | |
| ARATH\|TAIR = | Q9LQC0 | Heme oxygenase | HEME | oxygenase | Arabidopsis | A0A1S4A289 | LOC107792958 |
| locus = 2016635\| | | 4, chloroplastic | OXYGENASE 4, | | thaliana | | |
| UniProtKB = | | HO4 | CHLOROPLASTIC | | | A0A1S4CGA1 | LOC107818591 |
| Q9LQC0 | | | (PTHR35703: SF4) | | | | |
| ARATH\|TAIR = | P42043 | Ferrochelatase-1, | FERRO- | lyase | Arabidopsis | A0A1S3YBU7 | LOC107774460 |
| locus = 2180642\| | | chloroplastic/ | CHELATASE-1, | | thaliana | | |
| UniProtKB = | | mitochondrial | CHLOROPLASTIC/ | | | | |
| P42043 | | FC1 | MITOCHONDRIAL | | | A0A1S4CDJ6 | LOC107817912 |
| | | | (PTHR11108: SF4) | | | | |
| ARATH\|TAIR = | Q9C9L4 | Heme oxygenase | HEME | oxygenase | Arabidopsis | A0A1S4A289 | LOC107792958 |
| locus = 2205045\| | | 3, chloroplastic | OXYGENASE 1, | | thaliana | | |
| UniProtKB= | | HO3 | CHLOROPLASTIC- | | | A0A1S4CGA1 | LOC107818591 |
| Q9C9L4 | | | RELATED | | | | |
| | | | (PTHR35703: SF2) | | | | |
| ARATH\| | O48722 | Probable inactive | INACTIVE HEME | oxygenase | Arabidopsis | A0A1S3YTN1 | LOC107779560 |
| EnsemblGenome = | | heme oxygenase 2. | OXYGENASE 2, | | thaliana | | |
| AT2G26550\| | | chloroplastic | CHLOROPLASTIC- | | | | |
| UniProIKB = | | HO2 | RELATED | | | A0A1S3Y814 | LOC107773431 |
| O48722 | | | (PTHR35703: SF1) | | | | |

Ortholog mapping performed using - PANTHER (Protein ANalysis THrough Evolutionary Relationships) Version 16.0 (released 2020 Dec. 1)

In certain embodiments, the heme biosynthesis pathway proteins share at least 60% sequence identity, at least 65% sequence identity, at least 70% sequence identity, at least 75% sequence identity, at least 80% sequence identity, at least 85% sequence identity, at least 90% sequence identity, at least 95% sequence identity, at least 96% sequence identity, at least 97% sequence identity, at least 98% sequence identity, or at least 99% sequence identity to the amino acid sequence of the corresponding wild-type heme-containing protein or fragments thereof that contain a heme-binding motif. In certain embodiments, the heme biosynthesis pathway proteins share at least 60% sequence identity, at least 65% sequence identity, at least 70% sequence identity, at least 75% sequence identity, at least 80% sequence identity, at least 85% sequence identity, at least 90% sequence identity, at least 95% sequence identity, at least 96% sequence identity, at least 97% sequence identity, at least 98% sequence identity, or at least 99% sequence identity to the proteins set forth in Table 2.

In certain embodiments, the transgenic plants and recombinant nucleic acid sequences as described herein comprise genes encoding magnesium chelatase enzymes and/or modified or variant or mutant magnesium chelatase enzymes. In certain embodiments, the transgenic plants comprise knockouts or knockdowns of one or more genes encoding magnesium chelatase enzymes. Magnesium chelatase enzymes can play a regulatory role in directing and controlling flux down various branches of tetrapyrrole metabolism (e.g. magnesium chelatase initiates the biosynthetic pathways for these pigments by inserting Mg2+ into the protoporphyrin macrocycle; for example see Adams et al., et al. Nat. Plants 6, 1491-1502 (2020)). In certain embodiments, the transgenic plants as described herein comprise modified, mutated, and/or knockouts or knockdowns of one or more genes encoding magnesium chelatase enzymes selected from the genes of Table 3 and/or Table 4.

TABLE 3
Mg chelatase genes

Arabidopsis thaliana
GENOMES UNCOUPLED 4 (GUN4 (AT3G59400, GenBank 825109))
P-loop containing nucleoside triphosphate hydrolases
superfamily protein
(CHLI1 (AT4G18480, GenBank 827580))
magnesium-chelatase subunit chlH (CHLH (AT5G13630,
GenBank 831207))
magnesium chelatase I2 (CHLI2 (AT5G45930,
GenBank 834633))
Nicotiana tabacum
magnesium-chelatase subunit ChlI (LOC107764312,
GenBank 107764312)
magnesium-chelatase subunit ChlH (LOC107802255,
GenBank 107802255)
magnesium-chelatase subunit ChlD (LOC107820629,
GenBank 107820629)
Lactuca sativa
magnesium-chelatase subunit ChlI (LOC111884594,
GenBank 111884594)
magnesium-chelatase subunit ChlH (LOC111879487,
GenBank 111879487;
LOC111879365, GenBank 111879365)
magnesium-chelatase subunit ChlD (LOC111911728,
GenBank 111911728)
Glycine max
magnesium chelatase subunit (CHLH) (GLYMA . . . 03G137000,
GenBank 548043))

TABLE 4
Mg chelatase genes

Lactuca sativa

| | Gene ID | Description | Location | Aliases |
|---|---|---|---|---|
| 3 | ID: 111911728 | magnesium-chelatase subunit ChlD, | Chromosome 2, | LSAT_2X11 |
| | | chloroplastic [Lactuca sativa] | NC_056624.1 | 1701 |
| | | | (189869542 . . . 189873753) | |
| 2 | ID: 111879487 | magnesium-chelatase subunit ChlH, | Chromosome 9, | LSAT_9X29 |
| | | chloroplastic [Lactuca sativa] | NC_056631.1 | 121 |
| | | | (32205093 . . . 32210227, | |
| | | | complement) | |
| 1 | ID: 111884594 | magnesium-chelatase subunit ChlI, | Chromosome 4, | LSAT_4X50 |
| | | chloroplastic [Lactuca sativa] | NC_056626.1 | 480 |
| | | | (75068962 . . . 75071359) | |
| 2 | ID: 111879365 | magnesium-chelatase subunit ChlH, | Chromosome 9, | LSAT_9X29 |
| | | chloroplastic [Lactuca sativa] | NC_056631.1 | 020 |
| | | | (32157905 . . . 32162732) | |
| | ID: 122197911 | magnesium-chelatase subunit ChlH, | Chromosome 5, | |
| | | chloroplastic-like [Lactuca sativa] | NC_056627.1 | |
| | | | (229321031 . . . 229321628) | |

Arabidopsis thaliana

| | Locus | Description |
|---|---|---|
| 1 | AT3G59400 | Other names: GENOMES UNCOUPLED 4, GUN4 |
| | | GUN |
| 2 | AT4G18480 | Other names: CH-42, CH42, CHLI1, CHLI-1, CHLI1, |
| | | CHLORINA 42, LOST1, LOW TEMPERATURE WITH |
| | | OPEN-STOMATA 1 |
| 3 | AT5G13630 | Other names: ABA-BINDING PROTEIN, ABAR, CCH, |
| | | CCH1, CHLH, CONDITIONAL CHLORINA, GENOMES |
| | | UNCOUPLED 5, GUN5, H SUBUNIT OF MG-CHELATASE |
| 4 | AT5G45930 | Other names: CHL I2, CHLI-2, CHLI2, MAGNESIUM |
| | | CHELATASE I2 |

Nicotiana tabacum

| | Gene ID | Description | Aliases |
|---|---|---|---|
| 1 | LOC107764312 | magnesium-chelatase subunit ChlI, chloroplastic | CHLI, su, |
| | ID: 107764312 | [Nicotiana tabacum (common tobacco)] | sus, sulfur |
| 2 | LOC107802255 | magnesium-chelatase subunit ChlH, chloroplastic | |
| | ID: 107802255 | [Nicotiana tabacum (common tobacco)] | |
| | LOC107791852 | magnesium-chelatase subunit ChlH, chloroplastic-like | ChlH |
| | ID: 107791852 | [Nicotiana tabacum (common tobacco)] | |
| | LOC107783891 | magnesium-chelatase subunit ChlI, chloroplastic-like | |
| | ID: 107783891 | [Nicotiana tabacum (common tobacco)] | |
| 3 | LOC107820629 | magnesium-chelatase subunit ChlD, chloroplastic | |
| | ID: 107820629 | [Nicotiana tabacum (common tobacco)] | |
| | LOC107804319 | magnesium-chelatase subunit ChlD, chloroplastic-like | |
| | ID: 107804319 | [Nicotiana tabacum (common tobacco)] | |
| | LOC107788874 | magnesium-chelatase subunit ChlD, chloroplastic-like | |
| | ID: 107788874 | [Nicotiana tabacum (common tobacco)] | |
| | LOC107813465 | magnesium-chelatase subunit ChlH, chloroplastic-like | |
| | ID: 107813465 | [Nicotiana tabacum (common tobacco)] | |
| | LOC107813659 | uncharacterized LOC107813659 | |
| | ID: 107813659 | [Nicotiana tabacum (common tobacco)] | |
| | D5265_RS02985 | phage protein [Enterobacter mori] | D526 |
| | ID: 64724472 | | |

Arabidopsis thaliana

| | Name/Gene | | | |
|---|---|---|---|---|
| | ID | Description | Location | Aliases |
| 3 | GUN5 | magnesium-chelatase subunit | Chromosome 5, | AT5G13630, ABA- |
| | ID: 831207 | chlH, chloroplast, putative/ | NC_003076.8 | BINDING PROTEIN, |
| | | Mg-protoporphyrin IX | (4387307 . . . 4392429, | ABAR, CCH, CCHI, |
| | | chelatase, putative (CHLH) | complement) | CHLH, CONDITIONAL |
| | | [Arabidopsis thaliana | | CHLORINA, GENOMES |
| | | (thale cress)] | | UNCOUPLED 5, H |
| | | | | SUBUNIT OF MG- |
| | | | | CHELATASE, T6I14.12 |
| 4 | CHLI2 | magnesium chelatase i2 | Chromosome 5, | AT5G45930, CHL I2, |
| | ID: 834633 | [Arabidopsis thaliana | NC_003076.8 | CHLI2, CHLI-2, |
| | | (thale cress)] | (18627950 . . . 18629697) | K15122.13, K15122_13, |
| | | | | MAGNESIUM |
| | | | | CHELATASE I2, |
| | | | | magnesium chelatase i2 |
| | CHLM | magnesium-protoporphyrin IX | Chromosome 4, | AT4G25080, F24A6.6, |
| | ID: 828611 | methyltransferase | NC_003075.7 | MGPIXMT, magnesium- |
| | | [Arabidopsis thaliana | (12876822 . . . 12878703) | protoporphyrin IX |
| | | (thale cress)] | | methyltransferase |
| 2 | CHLI1 | P-loop containing nucleoside | Chromosome 4, | AT4G18480, CH-42, |
| | ID: 827580 | triphosphate hydrolases | NC_003075.7 | CH42, CHLI1, CHLI-1, |
| | | superfamily protein | (10201672 . . . 10203469, | CHLORINA 42, |
| | | [Arabidopsis thaliana | complement) | F28J12.140, F28J12_140, |
| | | (thale cress)] | | LOST1, |
| | | | | PROTOPORPHYRIN-IX |
| | | | | MG-CHELATASE, low |
| | | | | temperature with open- |
| | | | | stomata l |
| | ALB1 | ALBINA 1 | Chromosome 1, | AT1G08520, ALB-IV, |
| | ID: 837374 | [Arabidopsis thaliana | NC_003070.9 | ALBINA 1, CHLD, |
| | | (thale cress)] | (2696381 . . . 2701022) | PDE166, PIGMENT |
| | | | | DEFECTIVE EMBRYO |
| | | | | 166, T27G7.20, T27G7_20, |
| | | | | V157 |
| 1 | GUN4 | protein GENOMES | Chromosome 3, | AT3G59400, GENOMES |
| | ID: 825109 | UNCOUPLED 4 | NC_003074.8 | UNCOUPLED 4 |
| | | [Arabidopsis thaliana | (21948501 . . . 21949796, | |
| | | (thale cress)] | complement) | |
| | upf3 | magnesium protoporphyrin | | VOLCADRAFT_76537 |
| | ID: 9622075 | chelatase [Volvox carteri f. | | |
| | | nagariensis] | | |
| | AT3G44510 | alpha/beta-Hydrolases | Chromosome 3, | AT3G44510 |
| | ID: 823576 | superfamily protein | NC_003074.8 | |
| | | [Arabidopsis thaliana | (16108100 . . . 16111316, | |
| | | (thale cress)] | complement) | |
| | AT3G52570 | alpha/beta-Hydrolases | Chromosome 3, | AT3G52570, F3C22.3 |
| | ID: 824423 | superfamily protein | NC_003074.8 | |
| | | [Arabidopsis thaliana | (19501130 . . . 19503027) | |
| | | (thale cress)] | | |
| | AT2G21860 | violaxanthin de-epoxidase-like | Chromosome 2, | AT2G21860, F7D8.18, |
| | ID: 816722 | protein | NC_003071.7 | F7D8_18 |
| | | [Arabidopsis thaliana | (9318122 . . . 9320024, | |
| | | (thale cress)] | complement) | |
| | ID: 3974662 | [Oryza sativa (Asian | | |
| | | cultivated rice)] | | |
| | ID: 3760028 | [Hordeum vulgare (barley)] | | |

Glycine max

| | Name/Gene | | | |
|---|---|---|---|---|
| | ID | Description | Location | Aliases |
| | LOC100816614 | magnesium-chelatase subunit ChlD, | Chromosome 11, | GLYMA_11 |
| | ID: 100816614 | chloroplastic | NC_038247.2 | G016000, |
| | | [Glycine max | (1113068 . . . 1123336, | ChlD1 |
| | | (soybean)] | complement) | |
| | LOC100775919 | magnesium-chelatase subunit ChlD, | Chromosome 1, | GLYMA_01 |
| | ID: 100775919 | chloroplastic | NC_016088.4 | G226700, |
| | | [Glycine max | (56605577 . . . 56615286) | ChlD2 |
| | | (soybean)] | | |
| | CHLI | Mg-protoporphyrin IX chelatase | Chromosome 13, | GLYMA_13 |
| | ID: 100784256 | subunit ChlI | NC_038249.2 | G232500, |
| | | [Glycine max | (33762111 . . . 33764560) | ChlI1a |
| | | (soybean)] | | |
| | LOC100815274 | magnesium-chelatase subunit ChlI, | Chromosome 15, | GLYMA_15 |
| | ID: 100815274 | chloroplastic | NC_038251.2 | G080200 |
| | | [Glycine max | (6141416 . . . 6145917, | ChlI1b |
| | | (soybean)] | complement) | |
| | LOC100806079 | magnesium-chelatase subunit ChlH, | Chromosome 10, | GLYMA_10 |
| | ID: 100806079 | chloroplastic | NC_038246.2 | G097800, |
| | | [Glycine max | (15092415 . . . 15098589, | ChlH3 |
| | | (soybean)] | complement) | |
| | LOC100801560 | magnesium-chelatase subunit ChlI, | Chromosome 13, | GLYMA_13 |
| | ID: 100801560 | chloroplastic | NC_038249.2 | G171800, |
| | | [Glycine max | (28005148 . . . 28007927) | ChlI2b |
| | | (soybean)] | | |
| | LOC100787735 | magnesium-chelatase subunit ChlH, | Chromosome 19, | GLYMA_19 |
| | ID: 100787735 | chloroplastic | NC_038255.2 | G139300, |
| | | [Glycine max | (40503407 . . . 40510016) | ChlH2 |
| | | (soybean)] | | |
| | LOC100781940 | magnesium-chelatase subunit ChlI, | Chromosome 7, | GLYMA_07 |
| | ID: 100781940 | chloroplastic | NC_038243.2 | G204300, |
| | | [Glycine max | (37655434 . . . 37657913, | ChlI2a |
| | | (soybean)] | complement) | |
| 1 | CHLH | magnesium chelatase subunit | Chromosome 3, | GLYMA_03 |
| | ID: 548043 | [Glycine max | NC_016090.4 | G137000, |
| | | (soybean)] | (36476787 . . . 36483353) | CHlH1 |
| | LOC102664239 | magnesium-chelatase subunit ChlH, | Chromosome 20, | GLYMA_20 |
| | ID: 102664239 | chloroplastic | NC_038256.2 | G073800 |
| | | [Glycine max | (26026524 . . . 26027051, | |
| | | (soybean)] | complement) | |
| | LOC102669869 | magnesium-chelatase subunit ChlH, | Chromosome 20, | |
| | ID: 102669869 | chloroplastic-like | NC_038256.2 | |
| | | [Glycine max | (29845738 . . . 29846172, | |
| | | (soybean)] | complement) | |
| | LOC113000430 | magnesium-chelatase subunit ChlH, | Chromosome 19, | |
| | ID: 113000430 | chloroplastic-like | NC_038255.2 | |
| | | [Glycine max | (40526789 . . . 40528039) | |
| | | (soybean)] | | |

In certain embodiments, the recombinant nucleic acid sequences as disclosed herein include one or more genes that have been codon-optimized for the plant in which the recombinant nucleic acid sequences is to be expressed. For example, a recombinant nucleic acid sequences or construct to be expressed in a plant can have all or parts of its sequence codon-optimized for expression in a plant by methods known in the art.

The term “operably linked” as used herein refers to a functional linkage between a promoter sequence and a gene of interest, such that the promoter sequence is able to initiate transcription of the gene of interest. In certain embodiments, the one or more recombinant nucleic acid sequences may comprise a promoter suitable for expression in plants, a plant tissue or plant cell specific promoter, or an inducible promoter.

As used herein, “targeting sequences for homologous recombination in the transgenic plant chloroplast DNA” “flanking regions” or “flanking sequences” can be used interchangeably and refer to any sequences that are necessary for homologous recombination and integration of one or more transgene cassettes into a plastid genome (plastome) of a given plant at a specific position. In certain embodiments, the one or more flanking region(s) can include, but are not limited to complete homology to a sequence in the plastid genome of plant species (for example, trnl/trnA, rbcL/accD, trnfM-trnG, trnV/rps12, trnN-trnR or yef3-trnS). In certain embodiments, the sequence is trnl/trnA.

As used herein, the terms “selectable marker.” “selectable marker gene” or “reporter gene” can be used interchangeably and refer to any gene that confers a phenotype on a cell in which it is expressed to facilitate the identification and/or selection of cells that are transfected or transformed with a nucleic acid construct of the disclosure. Selectable marker genes enable the identification of a successful transfer of the one or more recombinant nucleic acid molecules. Suitable markers may be selected from markers, for example, that confer antibiotic or herbicide resistance, that introduce a new metabolic trait or that allow visual selection. Non-limiting examples of selectable marker genes can include, but is not limited to genes conferring resistance to antibiotics (such as Neomycin Phosphotransferase (nptll) that phosphorylates neomycin and kanamycin, Aminoglycoside 3′-Phosphotransferase (aphA6) that confers resistance to kanamycin or hpt, phosphorylating hygromycin, or genes conferring resistance to, for example, bleomycin, streptomycin, tetracycline, chloramphenicol, ampicillin, gentamycin, geneticin (G418), spectinomycin or blasticidin), to herbicides (for example, aroA or gox providing resistance against glyphosate, or resistance to phosphinothricin in plants by expression of the bialaphos resistance (BAR) or phosphinothricin acetyltransferase (PAT) genes, or the genes conferring resistance to, for example, imidazolinone, phosphinothricin or sulfonylurea), or genes that provide a metabolic trait (such as manA that allows plants to use mannose as sole carbon source or xylose isomerase for the utilization of xylose, or anti-nutritive markers such as the resistance to 2-deoxyglucose). Expression of visual marker genes results in the formation of color (for example, beta-glucuronidase, GUS or beta-galactosidase with its colored substrates, for example, X-Gal), luminescence (such as the luciferin/luciferase system) or fluorescence (Green Fluorescent Protein, GFP, and derivatives thereof). In some embodiments, the selection marker is streptomycin. In certain embodiments, the selection marker is spectinomycin. In some embodiments, the selection marker can be removable, for example, after successful selection of transgenic plants. In certain embodiments, the transgenic plants disclosed herein comprise one or more different selectable markers. For example, the transgenic plants can comprise two, three, four or five different selectable markers.

In some embodiments, the recombinant nucleic acid sequences disclosed herein comprise tagging the myoglobin protein. Protein tags can be used to purify proteins for which no protein-specific antibody exists, and can be fused to a protein at either the N-terminas or C-terminus of the protein using the recombinant nucleic acid sequences. In certain embodiments, protein tags can include, but are not limited to, His (polyhistidine, for example, 6x-His; (HHHHHH; SEQ ID NO:36), FLAG (DYKDDDDK: SEQ ID NO:37), glutathione S-transferase (GST), CMB3, and Myc. Tag-specific capture reagents such as affinity resins or antibody-linked beads are available to assist in the isolation and purification of proteins linked with at least one tag. In some embodiments, protein tags are removable by chemical agents or by enzymatic means.

### Isolation of Myoglobin

The transgenic plants and methods as disclosed herein are used to produce myoglobin in the transgenic plants, from which the myoglobin is then isolated. The term “isolated” as used herein, refers to molecules (e.g., myoglobin proteins) that are substantially separated or purified away from other molecules of the same type (e.g., other polypeptides) with which the molecule is normally associated in the cell of the organism in which the molecule naturally occurs. The term “substantially purified.” as used herein, refers to a molecule that is separated from other molecules normally associated with it in its native state. A substantially purified molecule may be, for example, at least 75% free, at least 80% free, at least 85% free, at least 90% free, at least 95% free, at least 96% free, at least 97% free, at least 98% free, or at least 99% free from other molecules besides a solvent present in a mixture. The term “substantially purified” does not refer to molecules present in their native state.

In certain embodiments, the myoglobin protein can be isolated from the transgenic plants based on molecular weight, for example, by size exclusion chromatography, ultrafiltration through membranes, or density centrifugation. In some embodiments, myoglobin proteins can be separated based on their surface charge, for example, by isoelectric precipitation, anion exchange chromatography, or cation exchange chromatography. Myoglobin proteins also can be separated on the basis of their solubility, for example, by ammonium sulfate precipitation, isoelectric precipitation, surfactants, detergents or solvent extraction. Myoglobin proteins also can be separated by their affinity to another molecule, using, for example, hydrophobic interaction chromatography, reactive dyes, or hydroxyapatite.

The invention will be further described in the following examples, which do not limit the scope of the invention described in the claims.

### EXAMPLES

The Examples that follow are illustrative of specific embodiments of the invention and various uses thereof. They are set forth for explanatory purposes only, and are not to be taken as limiting the invention. The following describes a cost-efficient animal meat protein production system for producing myoglobin and testing its characteristic under physiological conditions. The compositions and methods described herein provide transplastomic technology which enables chloroplasts to generate high levels of recombinant foreign proteins within plant leaves. This technology offers minimal risk of human pathogens and is free from a sterile laboratory environment for growth facilities, eliminates complex downstream processing such as protein purification steps, and abolishes cold chains.

### Chloroplast Transformation Construct Design and Gene Synthesis

1. In silico analysis to simulate plasmid construction
1.1. Deliver chloroplast transformation vectors from Addgene database.
1.2. Sequence genetic information of Mb gene and the other elements from gene databases.
1.3. In silico plasmid vector construction (using Genious software)

2. Molecular cloning
2.1. Codon optimization service of the constructs
2.2. Codon optimized sequences of construct for custom DNA synthesis and assembly service
2.3. Check if all constructs are ready for transformation

### Plant Transformation and Achieving Homoplasmy

3. Preparation of the materials for plant chloroplast transformation
3.1. Prepare plants for transformation
3.2. Prepare the transformation grade plasmid DNA
3.3. Preparing growth media for transformant cultivation

4. Plant chloroplast transformation by particle bombardment method
4.1. Operate biolistic particle delivery system (vector DNA on gold nanoparticle)
4.2. Cultivate the bombarded leaves (tissues on RMOP medium)

5. Chloroplast transformed (transplastomic) plant regeneration

### Primary Selection

5.1. Cultivate leaf disks on selection medium for callus and primary shoot induction
5.2. Assess the transformation efficiency by observing the callus appeared (˜two weeks after transformation)

6. Achieving homoplasmy

### Secondary Selection

6.1. Cultivate the leaf disks of the primary shoot onto selection medium for secondary shoot induction
6.2. Validate the gene insertion by PCR
6.3. Test the homoplasmy/heteroplasmy states by southern blotting analysis
6.4. Validate if the transformants possess the transgenes and how far to reach homoplasmy. Perform a third
6.5. Regeneration round, if necessary.
6.6. Transfer the leaf disks of secondary shoot onto selection medium for tertiary shoot induction

### Third Round Selection (if Necessary)

7. Root induction and seed production of transplastomic plants
7.1. Cultivate the positive transformants on the MS medium for root induction
7.2. Grow the rooted transformants in soil for seed production
7.3. Grow plants for pollination
7.4. Cross-pollinate transformants with pollen from non-transplastomic plants used for transformation
7.5. Harvest & test the seeds with MS selection medium for homoplasmicity
7.6. Confirmation of genotype of transplasmic tobacco plants

### Bovine Mb Protein Extraction and Purification

8. Optimization of the protein extraction and purification methods
8.1. Grow transplastomic plants in the growth chamber and green house
8.2. Optimization of protein extraction (e.g. organ type, growth stage)
8.3. Test protein accumulation level by Immunoblotting
8.4. Determine the optimal conditions for recombinant protein extraction
8.5. Purify the recombinant protein by using chromatographic methods
8.6. Determine the optimal conditions for recombinant protein purification

9. Examine the characteristics of the plant-produced Mb proteins
9.1. Examine characteristics of purified recombinant proteins in the native condition by native MS analysis
9.2. Evaluate the primary structure of purified Mb protein by LC-MS/MS analysis
9.3. Assessment of quality and quantity of plant-produced Mb protein

### Methods

### Plant Materials and Growth Conditions

A Chlamydomonas reinhardti strain CC-1690 was used as a wild-type strain. A heterotrophic mutant line HT72 that is a psbH:: aadA knockout mutant (a deletion of the essential photosystem Il gene psbH) in the background of the wild-type strain CC-1690 was used as a recipient strain for chloroplast transformation. Chlamydomonas reinhardtii cells were grown mixotrophically on Tris-acetate-phosphate (TAP) media (Gorman and Levine, 1965) (Tris-HCl [2.42 g L-1], NH4 Cl [0.375 g L-1], MgSO4·7H2O [0.1 g L-1], CaCl₂), 2H2O [0.05 g L-1], K2 HPO4 [0.10g L-1], KH2 PO4 [0.05 g L-1] Hutner's trace elements (prepared according to Harris (1989); NaEDTA [50 g L-1], ZnSO4, 7H2O [22 g L-1], H3 BO3 [11.4 g L-1], MnCl2, 4H2O [5.06 g L-1], CoCl2, 6H2O [1.61 g L-1], CuSO4, 5H2O) [1.57 g L-1], (NH4)6 Mo7 O24, 4H2O [1.1 g L-1], FeSO4, 7H2O [4.99 g L-1]) 1 ml L-1, acetic acid [1 ml L-1], pH 7.0) or photoautotrophically in High Salt Minimal (HSM) medium (NH4 Cl [0.5 g L-1], MgSO4, 7H2O [0.246 g L-1], CaCl2, 2H2O [0.01 g L-1], K2 HPO4 [1.44 g L-1], KH2 PO4 [0,72 g L-1], Hutner's Trace Elements 1 ml L-1. pH 6.8) with shaking at 140 rpm, or grown on 2% (w/v) agar TAP plates or 2% (w/v) agar HSM plates at a light intensity of ˜2 μE m-2s-1(mixotrophically) or light intensity of 60 μE m-26-1 (photoautotrophically).

Nicotiana tabacum cv. Petit Havana was used as a wild-type line. For surface sterilization, tobacco seeds were placed in centrifuge tubes, sealed with gauze, and incubated in a vacuum for 6 hours in a desiccator together with a flask containing 50 ml, 12% (w/v) sodium hypochlorite solution mixed with 2 ml, of 37% HCl. After sterilization, seeds were sown on MS or RM plant maintenance medium. Nicotiana tabacum plants and tissue cultures were grown on RM plant maintenance medium (0.56% (w/v) agar Murashige and Skoog (MS) medium (Murashige and Skoog, 1962) containing 2% (w/v) sucrose) or RMOP shoot regeneration medium with or without appropriate antibiotics (500 mg/L. Spectinomycin or 500 mg/L Streptomycin). Regenerated shoots from transplastomic lines were rooted and propagated on the RM plant maintenance medium. Rooted homoplasmic plants were transferred to soil and grown to maturity in a growth chamber at 23° C. with a 16-hour photoperiod and a light intensity of 50 μE m-2s-1.

Lactuca sativa cv. Simpson Elite was used as a wild-type line. For surface sterilization, lettuce seeds were placed in centrifuge tubes, sealed with gauze, and incubated in a vacuum for 6 hours in a desiccator together with a flask containing 50 mL, 12% (w/v) sodium hypochlorite solution mixed with 2 mL of 37% HCL. After sterilization, seeds were sown on MS or RM plant maintenance medium. Lactuca sativa plants and tissue cultures were grown on RM plant maintenance medium (0.7% (w/v) agar, Murashige and Skoog (MS) medium (Murashige and Skoog, 1962) containing 3% (w/v) sucrose) or RMOP shoot regeneration medium with or without appropriate antibiotics (500 mg/L Spectinomycin or 500 mg/L Streptomycin). Regenerated shoots from transplastomic lines were rooted and propagated on the RM plant maintenance medium. Rooted homoplasmic plants were transferred to soil and grown to maturity in a growth chamber at 24° ° C. with a 16-hour photoperiod and a light intensity of 40 μE m-2s-1.

Glycine max L. Merr., cv Jack was used as a wild-type line and the embryogenic tissues from Glycine mar were initiated as described by Santarem and Finer (1999). Following the first induction, embryogenic tissues were transferred to FNL medium, derived from Samoylov et al. (1998) (Dufourmantel et al. 2004). Embryogenic calli were maintained on FNL medium with or without f 200 mg/L, spectinomycin. Calli were converted into embryos on the medium described by Finer and McMullen (1991), containing 150 mg/l of spectinomycin. Embryos were transferred for germination to 0.7% (w/v) agar, Murashige and Skoog MS medium (Murashige and Skoog. 1962) at half ionic strength, containing 1.5% (w/v) of saccharose, 150 mg/L Spectinomycin. Regenerated shoots from transplastomic lines were rooted and propagated on the RM plant maintenance medium. Rooted homoplasmic plants were transferred to soil and grown to maturity in a growth chamber at 28° C. with a 16-hour photoperiod and a light intensity of 30 μE m-2s-1 (Finer et al., Plant Cell Tiss Organ Cult 15:125-36 (1988).

### Chloroplast Transformation Vector Construction

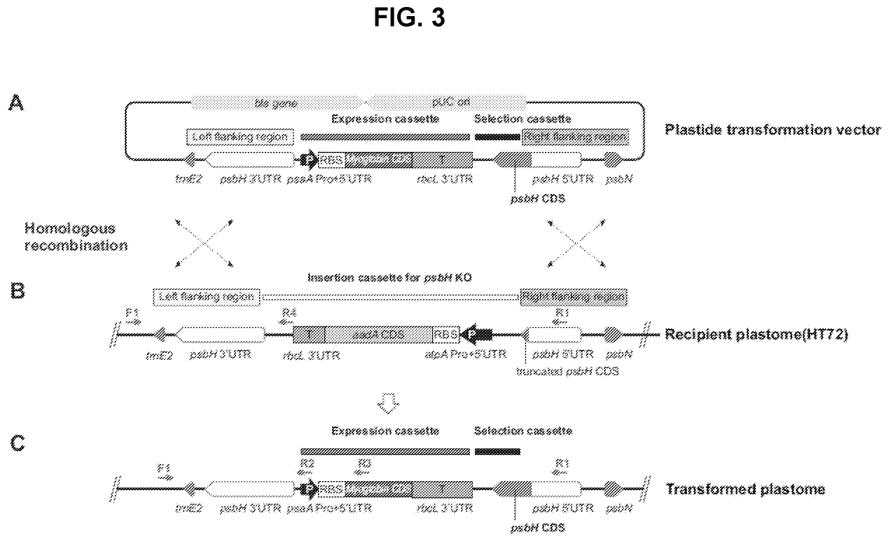

Constructions of expression cassettes and vectors used for Chlamydomonas reinhardtii are illustrated in FIG. 3. All of the coding sequences of the myoglobin genes (Bos taurus, Sus scrofa and Thunnus thynnus) were optimized for expression in Chlamydomonas reinhardtii chloroplast and chemically synthesized with desired flanking regions to facilitate molecular cloning. The synthetic genes coding for the Myoglobin were assembled with Psa. 4 promoter and rbel. 3′ UTR as an expression cassette flanked by homologous flanking sequences that consist of plastome sequences that include psbH as a selectable marker.

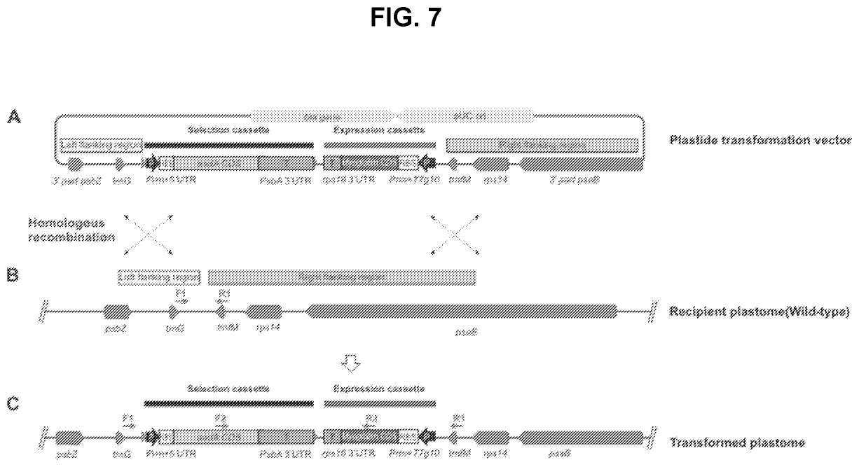

Constructions of expression cassettes and vectors used for Nicotiana tabacum are illustrated in FIG. 7. All of the coding sequences of the Myoglobin genes (Bos taurus, Sus scrofa and Thunnus thynnus) are optimized for expression in Nicotiana tabacum chloroplast and chemically synthesized with desired flanking regions to facilitate molecular cloning. The synthetic genes coding for the Myoglobin were assembled with rrn promoter followed by the bacteriophage T7 gene 10 leader sequence and rps 16 3′-UTR as an expression cassette and cloned in the multiple cloning site of the destination vector harboring addA gene encoding aminoglycoside 3′-adenyltransferase as a selectable marker conferring resistance to spectinomycin and streptomycin fused with rm promoter and PsbA 3′UTR flanked with loxP sites to facilitate selectable marker gene removal by Cre-mediated site-specific recombination (Corneille et al., 2001; Hajdukiewicz et al., 2001) and homologous flanking sequences that consist of plastome sequences spanning between the trn/M and trnG genes in the spacer region.

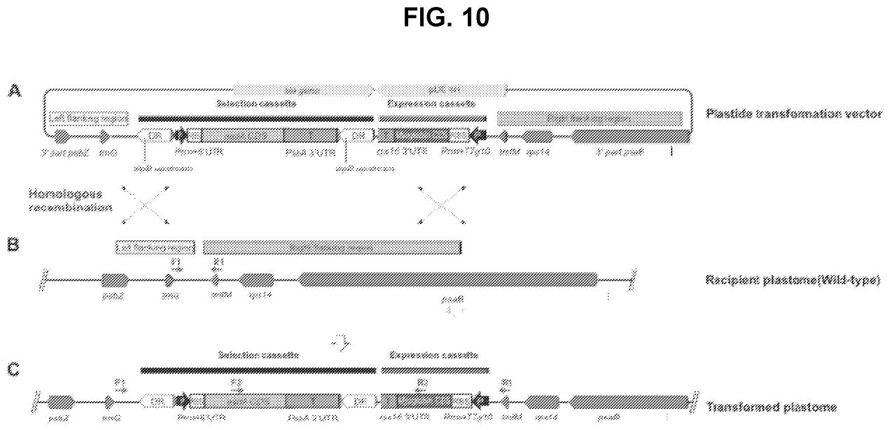

Constructions of expression cassettes and vectors used for Lactuca sativa are illustrated in FIG. 10. All of the coding sequences of the Myoglobin genes (Bos taurus, Sus scrofa and Thunnus thynnus) were optimized for expression in Loctuca sativa chloroplast and chemically synthesized with desired flanking regions to facilitate molecular cloning. The synthetic genes coding for the Myoglobin were assembled with rm promoter followed by the bacteriophage T7 gene 10 leader sequence and rps16 3′-UTR as an expression cassette and cloned in the multiple cloning site of the destination vector harboring addA gene encoding aminoglycoside 3′-adenyltransferase as a selectable marker conferring resistance to spectinomycin and streptomycin fused with rrn promoter and PsbA 3′UTR flanked with loxP sites to facilitate selectable marker gene removal by Cre-mediated site-specific recombination and direct repeats consist of the upstream region of Nicotiana tabacum atpB gene for homologous recombination to loop out the selectable marker gene and flanking sequences that consist of plastome sequences spanning between the trnfM and trnG genes in the spacer region.

### Biolistic Gene Delivery for Chloroplast Transformation

Plastid transformation was performed by biolistic transformation (Svab and Maliga, 1993). Plasmid DNA-loaded gold particles (0.6 μm diameter) were shot with a helium-driven particle gun (PDS1000He, Bio-Rad, Munich, Germany) into the cells of young plant leaves. Primary transformants were selected on spectinomycin-containing (500 mg/L) regeneration medium (RMOP). To eliminate lines with spontaneous mutations leading to antibiotic resistance, double resistance tests on a medium containing spectinomycin (500 mg/L) and streptomycin (500 mg/L) were performed. To obtain homoplasmic transplastomic lines, plants were subjected to 2-4 additional rounds of regeneration on the RMOP medium with spectinomycin.

### Isolation of Nucleic Acids

To investigate transgene integration in C. reinhardtii chloroplast genome, the C. reinhardtii cells were isolated from the single colonies on 2% (w/v) agar HSM plate and the genomic DNA were extracted in 5% Chelex 100 resin solution by heating at 95ºC for 10 min. The resultants were placed on ice to settle the resins to the bottom and the supernatants were used for polymerase chain reaction (PCR).

To investigate transgene integration in the N. tabacum chloroplast genome, the N. tabacum leaves were harvested from TO tobacco plants and immediately flash-frozen in liquid nitrogen. Total genomic DNA was extracted using a DNeasy plant mini kit (QIAGEN). Purity of the DNA extraction was assayed by measuring the spectrophotometric absorbance at 260 nm and 280 nm.

To investigate transgene integration in the L. sativa chloroplast genome, the L. sativa leaves are harvested from TO lettuce plants and immediately flash-frozen in liquid nitrogen. Total genomic DNA was extracted using a DNeasy plant mini kit (QIAGEN). Purity of the DNA extraction was assayed by measuring the spectrophotometric absorbance at 260 nm and 280 nm.

To investigate transgene integration in the G. max chloroplast genome, the G. max tissues are harvested from TO soybean plants and immediately flash-frozen in liquid nitrogen. Total genomic DNA was extracted using a DNeasy plant mini kit (QIAGEN). Purity of the DNA extraction was assayed by measuring the spectrophotometric absorbance at 260 nm and 280 nm.

### PCR and DNA Sequencing

PCR reactions were carried out using Q5® High-Fidelity DNA Polymerase (NEB) according to the manufacturer's instructions. PCR products were visualized following electrophoresis in a 0.8-1% agarose gel containing ethidium bromide.

PCR products were sequenced following either gel purification of the desired band using a Qiaquick Gel Extraction Kit (QIAGEN) or primer removal using a GeneJET PCR Purification Kit (Thermo Scientific) according to the manufacturer's instructions. Sangar sequencing was employed for DNA sequencing provided by Azenta Life Sciences.

### Transformation, Selection and Regeneration of Transplastomic Soybean Plants

Tissues were bombarded as described by Santarem and Finer (1999) using a helium-driven particle gun (PDS1000He. Bio-Rad, Munich, Germany). Fifteen-20 embryogenic calli were bombarded, on both sides, using plasmid DNA-loaded gold particles (0.6 μm diameter). Two days after bombardment, calli were cut into very small pieces (˜1.5-2 mm diameter) and transferred to a fresh FNL medium containing 200 mg/L of spectinomycin (or 300 mg/L of spectinomycin for the second round). Calli were transferred onto a fresh selection medium every fifteen days. The putative transformants were amplified in a SBP6 liquid medium with 150 mg/L of spectinomycin (Finer and Nagasawa, 1988). Calli were converted into embryos using the medium described by Finer and McMullen (1991), containing 150 mg/L of spectinomycin. After ˜2 months on this medium, embryos were dessicated for 2 days and then transferred for germination to MS medium (Murashige and Skoog, 1962) at half ionic strength, containing 15 g/L saccharose, 150 mg/L spectinomycin and 7 g/L, phytagar, pH 5.7. When young plants were well developed, they were transferred into soil for a 10-15 days acclimatisation period, before being transferred into the greenhouse for development and seed production. To test the transgene transmission to the progeny, seeds were sown into a MS medium with half ionic strength containing 15 g/L saccharose and 500 mg/L spectinomycin.

### Example 1: Generation of Chloroplast Transgenic Nicotiana tabacum (Tobacco) Plants Expressing Bos taurus Myoglobin (Bovine Mb), Sus scrofa Myoglobin (Porcine Mb), or Thunnus Thynnus (Tuna Mb)

The Myoglobin (Mb) gene of the domestic cow (Bos taurus; bovine), pig (Sus scrofa), and tuna (Thunnus thynnus) were chosen as exemplary myoglobin genes for expression in a transgenic plant. Bovine Mb was expressed in both a native (intact) recombinant protein and affinity-tagged recombinant protein, and CMB3, linked at either the N-terminus or C-terminus of the protein. The nucleic acid sequences coding bovine Mb, pig Mb, and tuna Mb proteins were codon optimized for expression in the Nicotiana tabacum (tobacco) chloroplast and synthesized using a gene synthesis service. To accelerate the maturation of Mb proteins, the co-factor heme was provided with the key enzymes in the native tobacco heme biosynthetic pathway (e.g. Ferrochelatase-2 A0AIS3YUH8) and was co-overexpressed with the bovine Mb, pig Mb, or tuna Mb gene.

Chloroplast transformation vectors were cloned with the myoglobin gene, and/or a gene cluster of myoglobin gene and heme biosynthesis pathway genes (it's possible that not all heme biosynthesis pathway genes are necessary for facilitating the maturation of the bovine, pig, or tuna Mb proteins by accelerating the native heme biosynthesis).

The plastid transformation vector can comprise two components: (1) an expression cassette comprising a gene of interest which is inserted between the plastid promoter and the plastid terminator, followed by a selection marker gene which is inserted between the plastid promoter and the plastid terminator, and (2) a targeting sequence for homologous recombination in the host plant plastid genome.

Wild-type tobacco plants were used for transformation with the plastid transformation vector comprising the bovine, pig or tuna Mb genes. Tobacco chloroplast transformation was effectuated by the particle bombardment method (Svab and Maliga, 1993; Lu et al. 2006; Scotti & Cardi, 2012). Briefly, plasmid DNA was coated onto gold beads and two-week-old tobacco seedlings were bombarded with DNA-coated beads. Leaves from bombarded seedlings were cultured on selection medium containing an appropriate antibiotic for 2-3 weeks. Newly generated shoots (primary shoots) were cut into pieces and transferred to freshly prepared selection medium. Secondary shoots were screened on MS medium containing an appropriate antibiotic for rooting. Leaves from rooted plants were subjected to PCR testing for insertion of the bovine Mb gene at the anticipated site in the chloroplast genome and southern blotting for verifying homoplasmy. Heteroplasmic transformants were subjected to further rounds of tissue culture on selection media to obtain homoplasmic transformants. Homoplasmic transformants are transferred to pots and grown in a greenhouse to produce seed.

Isolation and purification of bovine Mb from the transgenic tobacco plants —Tobacco seeds from the chloroplast transformed tobacco plants are sown in soil in a greenhouse and/or a field. Leaves of 4-11 weeks old tobacco plants are harvested and the crude proteins are extracted by homogenization in an appropriate buffer. Cell debris were removed by centrifugation and the protein extract is buffer exchanged using a molecular weight cut-off (MWCO) filter. Following the buffer exchange. Mb is purified by anion exchange chromatography using 5 ml Hitrap HP Q-Sepharose columns, with up to three columns connected in series, operated by a Biologic LP Chromatography System (Carlsson et al., 2020). The Mb fractions are then collected based on visible color (unique for heme) and chromatogram data. If necessary, the fraction is further purified on an appropriate affinity purification column.

Evaluation of quantity and quality of the tobacco plant-based bovine Mb and comparison to animal bovine Mb—The expression efficiency of the recombinant Mb protein is compared to the total soluble protein (TSP) by immunoblot analysis using commercially available anti-Mb antibodies. TSP concentration is measured using a standard Bradford assay and a concentration course of commercially available bovine Mb as a control. The quality of the recombinant bovine Mb protein is evaluated by two criteria: (a) if the primary structure (e.g. the amino acid sequence and post-translational modification) is identical to the native bovine Mb; and (b) if the recombinant Mb is incorporated into heme with the expected affinity. To evaluate (a), Liquid chromatography-mass spectrometry (LC-MS/MS) analysis is employed for sequencing the recombinant protein. To evaluate (b), native MS analysis is used, which allows the analysis of intact protein assemblies under non-denaturing conditions which provides in-depth structural characterization of protein properties such as solubility, molecular weight, folding, assembly state and stability. Topological arrangements are also employed.

### Example 2: Generation of Chloroplast Transgenic Lactuca sativa (Lettuce) Plants Expressing Bos taurus Myoglobin (Bovine Mb), Sus scrofa Myoglobin (Porcine Mb), or Thunnus Thynnus (Tuna Mb)

The Myoglobin (Mb) gene of the domestic cow (Bos taurus; bovine), pig (Sus scrofa), and tuna (Thunnus thynnus) were chosen as exemplary myoglobin genes for expression in a transgenic plant. Bovine Mb was expressed in both a native (intact) recombinant protein and affinity-tagged recombinant protein (e.g. cleavable 6x His tag, glutathione S—transferase (GST)) and CMB3, linked at either the N-terminus or C-terminus of the protein. The nucleic acid sequences coding bovine Mb, pig Mb, and tuna Mb proteins were codon optimized for expression in the Lactuca sativa chloroplast and synthesized using a gene synthesis service. To accelerate the maturation of Mb proteins, the co-factor heme was provided with the key enzymes in the native lettuce heme biosynthesis pathway (e.g. Ferrochelatase-2; Loc111894117) and was co-overexpressed with the bovine Mb, pig Mb, or tuna Mb gene.

Chloroplast transformation vectors were cloned with the myoglobin gene, and/or a gene cluster of myoglobin gene and heme biosynthesis pathway genes (it's possible that not all heme biosynthesis pathway genes are necessary for facilitating the maturation of the bovine, pig, or tuna Mb proteins by accelerating the native heme biosynthesis).

The plastid transformation vector can comprise two components: (1) an expression cassette comprising a gene of interest which is inserted between the plastid promoter and the plastid terminator, followed by a selection marker gene which is inserted between the plastid promoter and the plastid terminator that is flanked by can be loop-out to remove, and (2) a targeting sequence for homologous recombination in the host plant plastid genome.

Lettuce plants were used for transformation with the plastid transformation vector comprising the bovine, pig or tuna Mb genes. Lettuce chloroplast transformation was effectuated by the particle bombardment method (Svab and Maliga, 1993; Lu et al. 2006; Scotti & Cardi, 2012). Briefly, plasmid DNA was coated onto gold beads and two-week-old lettuce seedlings were bombarded with DNA-coated beads. Leaves from bombarded seedlings were cultured on selection medium containing an appropriate antibiotic for 2-3 weeks. Newly generated shoots (primary shoots) were cut into pieces and transferred to freshly prepared selection medium. Secondary shoots were screened on MS medium containing an appropriate antibiotic for rooting. Leaves from rooted plants were subjected to PCR testing for insertion of the bovine Mb gene at the anticipated site in the chloroplast genome and southern blotting for verifying homoplasmy. Heteroplasmic transformants were subjected to further rounds of tissue culture on selection media to obtain homoplasmic transformants. Homoplasmic transformants are transferred to pots and grown in a greenhouse to produce seed.

Isolation and purification of bovine Mb from the transgenic lettuce plants —Lettuce seeds from the chloroplast transformed lettuce plants are sown in soil in a greenhouse and/or a field. Leaves of 4-11 weeks old lettuce plants are harvested and the crude proteins are extracted by homogenization in an appropriate buffer. Cell debris are removed by centrifugation and the protein extract is buffer exchanged using a molecular weight cut-off (MWCO) filter. Following the buffer exchange, Mb is purified by anion exchange chromatography using 5 ml Hitrap HP Q-Sepharose columns, with up to three columns connected in series, operated by a Biologic LP Chromatography System (Carlsson et al., 2020). The Mb fractions are then collected $2 based on visible color (unique for heme) and chromatogram data. If necessary, the fraction is further purified on an appropriate affinity purification column.

Evaluation of quantity and quality of the lettuce plant-based bovine Mb and comparison to animal bovine Mb—The expression efficiency of the recombinant Mb protein is compared to the total soluble protein (TSP) by immunoblot analysis using commercially available anti-Mb antibodies. TSP concentration is measured using a standard Bradford assay and a concentration course of commercially available bovine Mb as a control. The quality of the recombinant bovine Mb protein is evaluated by two criteria: (a) if the primary structure (e.g. the amino acid sequence and post-translational modification) is identical to the native bovine Mb; and (b) if the recombinant Mb is incorporated into heme with the expected affinity. To evaluate (a), Liquid chromatography-mass spectrometry (LC-MS/MS) analysis is employed for sequencing the recombinant protein. To evaluate (b), native MS analysis is used, which allows the analysis of intact protein assemblies under non-denaturing conditions which provides in-depth structural characterization of protein properties such as solubility, molecular weight, folding, assembly state and stability. Topological arrangements are also employed.

### Example 3: Generation of Chloroplast Transgenic Glycine max (Soybean) Plants Expressing Bos taurus Myoglobin (Bovine Mb), Sus scrofa Myoglobin (Porcine Mb), or Thunnus Thynnus (Tuna Mb)

The Myoglobin (Mb) gene of the domestic cow (Bos taurus; bovine), pig (Sus scrofa), and tuna (Thunnus thynnus) are chosen as exemplary myoglobin genes for expression in a transgenic plant. Bovine Mb is expressed in both a native (intact) recombinant protein and affinity-tagged recombinant protein (e.g. cleavable 6x His tag, glutathione S—transferase (GST)) and CMB3, linked at either the N-terminus or C-terminus of the protein. The nucleic acid sequences coding bovine Mb, pig Mb, and tuna Mb proteins are codon optimized for expression in the Glycine max chloroplast and synthesized asing a gene synthesis service. To accelerate the maturation of Mb proteins, the co-factor heme is provided with the key enzymes in the native soybean heme biosynthesis pathway (e.g. a Ferrochelatase-2 enzyme) and is co-overexpressed with the bovine Mb, pig Mb, or tuna Mb gene.

Chloroplast transformation vectors are cloned with the myoglobin gene, and/or a gene cluster of myoglobin gene and heme biosynthesis pathway genes (it's possible that not all $3 heme biosynthesis pathway genes are necessary for facilitating the maturation of the bovine, pig, or tuna Mb proteins by accelerating the native heme biosynthesis).

The plastid transformation vector can comprise two components: (1) an expression cassette comprising a gene of interest which is inserted between the plastid promoter and the plastid terminator, followed by a selection marker gene which is inserted between the plastid promoter and the plastid terminator, and (2) a targeting sequence for homologous recombination in the host plant plastid genome.

Soybean plants are used for transformation with the plastid transformation vector comprising the bovine, pig or tuna Mb genes. Soybean chloroplast transformation are effectuated by the particle bombardment method (Svab and Maliga. 1993; Dufourmantel et al. 2004; Lu et al. 2006; Scotti & Cardi. 2012). Briefly, plasmid DNA is coated onto gold beads and soybean embryogenic calli were bombarded with DNA-coated beads. Embryogenic calli were cultured on selection medium containing an appropriate antibiotic for −8 weeks. The putative transformants were amplified in a SBP6 liquid medium with 150 mg/L spectinomycin (Finer and Nagasawa, 1988). Calli were converted into embryos using the medium described by Finer and McMullen (1991), containing 150 mg/L spectinomycin. After 2 months on this medium, embryos were dessicated for 2 days and then transferred for germination to MS medium at half ionic strength, containing 15 g/L, saccharose, 150 mg/L spectinomycin and 7 g/L phytagar, pH 5.7. Tissues from young plants are subjected to PCR testing for insertion of the Mb gene at the anticipated site in the chloroplast genome and southern blotting for verifying homoplasmy. Heteroplasmic transformants are subjected to further rounds of tissue culture on selection media to obtain homoplasmic transformants. Homoplasmic transformants are transferred to pots and grown in a greenhouse to produce seed.

Isolation and purification of bovine Mb from the transgenic soybean plants —Soybean seeds from the chloroplast transformed soybean plants are sown in soil in a greenhouse and/or a field. Tissues (e.g. seed, leaf and silique) of 4-16 weeks old soybean plants are harvested and the crude proteins are extracted by homogenization in an appropriate buffer. Cell debris are removed by centrifugation and the protein extract is buffer exchanged using a molecular weight cut-off (MWCO) filter. Following the buffer exchange, Mb is purified by anion exchange chromatography using 5 ml Hitrap HP Q-Sepharose columns, with up to three columns connected in series, operated by a Biologic LP Chromatography System (Carlsson et al., 2020). The Mb fractions are then collected based on visible color (unique for heme) and chromatogram data. If necessary, the fraction is further purified on an appropriate affinity purification column.

Evaluation of quantity and quality of the soybean plant-based bovine Mb and comparison to animal bovine Mb—The expression efficiency of the recombinant Mb protein is compared to the total soluble protein (TSP) by immunoblot analysis using commercially available anti-Mb antibodies. TSP concentration is measured using a standard Bradford assay and a concentration course of commercially available bovine Mb as a control. The quality of the recombinant bovine Mb protein is evaluated by two criteria: (a) if the primary structure (e.g. the amino acid sequence and post-translational modification) is identical to the native bovine Mb; and (b) if the recombinant Mb is incorporated into heme with the expected affinity. To evaluate (a), Liquid chromatography-mass spectrometry (LC-MS/MS) analysis is employed for sequencing the recombinant protein. To evaluate (b), native MS analysis is used, which allows the analysis of intact protein assemblies under non-denaturing conditions which provides in-depth structural characterization of protein properties such as solubility, molecular weight, folding, assembly state and stability. Topological arrangements are also employed.

### REFERENCES

Gray, B. N., Ahner, B. A. & Hanson, M. R. High-level bacterial cellulase accumulation in chloroplast-transformed tobacco mediated by downstream box fusions. Biotechnol. Bioeng. 102, 1045-1054 (2009)
Svab. Z., & Maliga, P. A. L. (1993). High-frequency plastid transformation in tobacco by selection for a chimeric aadA gene. Proceedings of the National Academy of Sciences, 90(3), 913-917.
Lu X M., Yin W B., Hu Z. M. (2006) Chloroplast Transformation. In: Loyola-Vargas V. M., Vázquez-Flota F. (eds) Plant Cell Culture Protocols. Methods in Molecular Biology™, vol 318. Humana Press.
Scotti, N., & Cardi, T. (2012). Plastid transformation as an expression tool for plant-derived biopharmaceuticals. In Transgenic Plants (pp. 451-466). Humana Press.
Carlsson, M. L. R., Kanagarajan, S., Bülow, L. et al. Plant based production of myoglobin—a novel source of the muscle heme-protein. Sci Rep 10, 920 (2020).
Sainsbury F. (2020). Innovation in plant-based transient protein expression for infectious disease prevention and preparedness. Current opinion in biotechnology, 61, 110.
Schillberg, S., Raven, N., Spiegel, H., Rasche, S., & Buntru, M. (2019). Critical analysis of the commercial potential of plants for the production of recombinant proteins. Frontiers in plant science, 10, 720.
Daniell, H., Choun-S, L., Ming, Y. & Wan- J, C. (2016) Chloroplast genomes: diversity, evolution, and applications in genetic engineering. Genome Biol. 17, 134.
Adem, M., Beyene, D. & Feyissa, T. (2017) Recent achievements obtained by chloroplast transformation. Plant Methods 13, 30.
Fraser, R., Brown, P., Karr, J., Holz-Schietinger, C., Cohn, E. (2017) Method and compositions for affecting the flavor and aroma profile of consumables.
United States: U.S. Patent Office; U.S. Pat. No. 9,700,067 B2.
Hurrell, R., & Egli, I. (2010). Iron bioavailability and dietary reference values. The American journal of clinical nutrition, 91, 1461S-1467S.
Schachtsiek, J., & Stehle, F. (2019) Dataset on nicotine-free, nontransgenic tobacco (Nicotiana tabacum 1) edited by CRISPR-Cas9. Data in brief 26, 104395.
Schachtsiek J, Stehle F. Nicotine-free, nontransgenic tobacco (Nicotiana tabacum L.) edited by CRISPR-Cas9. Plant Biotechnol J. 2019 December; 17(12):2228-2230.
Chen, Po-Yen, Yung-Ting Tsai, and Kin-Ying To. “Construction and Evaluation of Chloroplast Expression Vectors in Higher Plants.” Genetic Transformation in Crops. IntechOpen, 2020. Vaface Y, Staniek A, Mancheno-Solano M, Warzecha H (2014) A Modular Cloning Toolbox for the Generation of Chloroplast Transformation Vectors. PLOS ONE 9(10): e110222.
Fraser, Rachel Z., et al. “Safety evaluation of soy leghemoglobin protein preparation derived from Pichia pastoris, intended for use as a flavor catalyst in plant-based meat.” International journal of toxicology 37.3 (2018): 241-262.
Woodson J D, Perez-Ruiz J M, Chory J. Heme synthesis by plastid ferrochelatase 1 regulates nuclear gene expression in plants. Curr Biol. 2011; 21(10):897-903.
Espinas, N. A., Kobayashi, K., Sato, Y., Mochizuki, N., Takahashi, K., Tanaka, R. and Masuda, T., 2016. Allocation of heme is differentially regulated by ferrochelatase isoforms in Arabidopsis cells. Frontiers in plant science, 7, p.1326.
Layer, G., Reichelt, J., Jahn, D. and Heinz, D. W. (2010), Structure and function of enzymes in heme biosynthesis. Protein Science, 19: 1137-1161.
Tanaka, R., Kobayashi, K., and Masuda, T. (2011). Tetrapyrrole metabolism in Arabidopsis thaliana. Arabidopsis Book 9:e0145.
Kobayashi, Koichi, and Tatsuru Masada. “Transcriptional regulation of tetrapyrrole biosynthesis in Arabidopsis thaliana.” Frontiers in plant science 7 (2016): 1811.
Richter A S, Banse C, Grimm B. The GluTR-binding protein is the heme-binding factor for feedback control of glutamyl-tRNA reductase. Elife. 2019;8:e46300. Published 2019 Jun 13.
Hey D. Ortega-Rodes P, Fan T. Schnurrer F. Brings L, Hedtke B, Grimm B. Transgenic Tobacco Lines Expressing Sense or Antisense FERROCHELATASE I RNA Show Modified Ferrochelatase Activity in Roots and Provide Experimental Evidence for Dual Localization of Ferrochelatase 1. Plant Cell Physiol. 2016 Dec:57(12):2576-2585. Papenbrock J, Mishra S,
Mock H P, Kruse E, Schmidt E K, Petersmann A, Braun H P, Grimm B. Impaired expression of the plastidic ferrochelatase by antisense RNA synthesis leads to a necrotic phenotype of transformed tobacco plants. Plant J. 2001 October; 28(1):41-50. Suzuki, T., Masuda, T., Singh, D.P.,
Tan, F. C., Tsuchiya, T., Shimada, H., Ohta, H., Smith, A. G. and Takamiya, K. J., 2002, Two types of ferrochelatase in photosynthetic and nonphotosynthetic tissues of cucumber; their difference in phylogeny, gene expression, and localization. Journal of Biological Chemistry, 277(7), pp. 4731-4737.
Yihe Yu, Po-Cheng Yu, Wan-Jung Chang, Keke Yu, and Choun-Sea Lin, “Plastid Transformation: How Does it Work? Can it Be Applied to Crops? What Can it Offer?” Int. J. Mol. Sci. 2020, 21(14), 4854;
Islam M R, Kwak J W, Lee J S, et al. Cost-effective production of tag-less recombinant protein in Nicotiana benthamiana. Plant Biotechnology Journal. 2019 June; 17(6): 1094-1105. Sadali N M,
Sowden R G, Ling Q, Jarvis R P. Differentiation of chromoplasts and other plastids in plants. Plant Cell Rep. 2019:38(7):803-818.
Liebers M, Grübler B, Chevalier F, Lerbs-Mache S, Merendino L, Blanvillain R and Pfannschmidt T (2017) Regulatory Shifts in Plastid Transcription Play a Key Role in Morphological Conversions of Plastids during Plant Development. Front. Plant Sci. 8:23. doi: 10.3389/fpls.2017.00023
Gorman, D S. and R P Levine. “Cytochrome f and plastocyanin: their sequence in the photosynthetic electron transport chain of Chlamydomonas reinhardi.” PNAS vol. 54,6 (1965); 1665-9.
Harris, E. H. (1989): The Chlamydomonas sourcebook: a comprehensive guide to biology and laboratory use. Academic Press, San Diego, 780pp.
Wannathong T, Waterhouse J C, Young R E, Economou C K, Purton S. New tools for chloroplast genetic engineering allow the synthesis of human growth hormone in the green alga Chlamydomonas reinhardtii. Appl Microbiol Biotechnol. 2016:100(12):5467-5477.
Zhou, F., Badillo-Corona, J. A., Karcher, D., Gonzalez-Rabade, N., Piepenburg, K., Borchers, A. M., Maloney, A. P., Kavanagh, T. A., Gray, J. C., & Bock, R. (2008). High-level expression of human immunodeficiency virus antigens from the tobacco and tomato plastid genomes. Plant biotechnology journal, 6(9), 897-913.
Neupert J, Karcher D, Bock R. Design of simple synthetic RNA thermometers for temperature-controlled gene expression in Escherichia coli. Nucleic Acids Res. 2008;36(19):e124.
Fan, J., Zheng, L., Bai, Y., Saroussi, S., & Grossman, A. R. (2017). Flocculation of Chlamydomonas reinhardtii with different phenotypic traits by metal cations and high pH. Frontiers in plant science, 8, 1997.

1. A transgenic plant, wherein the transgenic plant comprises at least one chloroplast with one or more recombinant nucleic acid sequences expressing a myoglobin gene encoding a myoglobin protein.

2. The transgenic plant of claim 1, wherein the one or more recombinant nucleic acid sequences is integrated into the chloroplast DNA of the transgenic plant, and/or wherein the one or more recombinant nucleic acid sequences is stably integrated into the chloroplast DNA of the transgenic plant.

3. The transgenic plant of claim 1, wherein the transgenic plant comprises at least about 10 copies, at least about 100 copies, at least about 1,000 copies, at least about 5,000 copies, at least about 10,000 copies, at least about 20,000 copies, at least about 30,000 copies, at least about 40,000 copies, or at least about 50,000 copies of the one or more recombinant nucleic acid sequences.

4. The transgenic plant of claim 1, wherein the one or more recombinant nucleic acid sequences further comprises:
(a) one or more selectable markers, wherein the one or more selectable markers are optionally removable;
(b) one or more genes encoding one or more enzymes in the heme biosynthesis pathway;
(c) one or more targeting sequences for homologous recombination in the host transgenic plant chloroplast DNA.

5. The transgenic plant of claim 1, wherein the transgenic plant is: (a) a stable, homoplasmic transformant; or (b) a stable heteroplasmic transformant.

6. The transgenic plant of claim 1, wherein the myoglobin gene is a bovine myoglobin gene (for example, bison, buffalo, cow, goat, sheep, or yak), an avian myoglobin gene (for example, chicken, duck, goose, guinea fowl, quail, pigeon, or turkey), a suine myoglobin gene (for example, boar or pig), or a fish myoglobin (for example, tuna, salmon, or eel).

7. The transgenic plant of claim 1, wherein the myoglobin gene is selected from Table 1 and/or wherein the myoglobin gene encodes a myoglobin protein selected from SEQ ID NO's 1-35.

8. The transgenic plant of claim 1, wherein the myoglobin gene comprises a codon-optimized myoglobin gene, and wherein the codon-optimized myoglobin gene is codon-optimized for expression in the transgenic plant.

9. The transgenic plant of claim 1, wherein the myoglobin gene is operably linked to at least one promoter.

10. The transgenic plant of claim 1, wherein the transgenic plant is a grass (for example, a barely, a corn, a maize, an oat, a silvergrass, a sugarcane, a rice, a rye, or a wheat), a legume (for example, an alfalfa, a bean, a chickpea, a clover, a lentil, a pea, or a peanut), a nightshade (for example, an eggplant, a pepper, a potato, a tobacco, or a tomato), an aster (for example, a lettuce, a chamomile, an artichoke, an endive, a lavender, a cotton, a sunflower), or an alga, a moss, or a liverwort.

11. The transgenic plant of claim 10, wherein the transgenic plant is an aster, and the aster is a lettuce plant (i.e., a Lactuca species).

12. The transgenic plant of claim 10, wherein the transgenic plant is a legume.

13. The transgenic plant of claim 10, wherein the transgenic plant is a nightshade, and the nightshade is a tobacco plant (i.e., a Nicotiana species).

14. The transgenic plant of claim 1, wherein the transgenic plant comprises a knock-down or knock-out of one or more genes encoding magnesium chelatase enzymes.

15. The transgenic plant of claim 1, wherein the myoglobin protein comprises at least about 0.1%, at least about 1.0%, at least about 10%, at least about 20%, at least about 30%, at least about 40%, at least about 50%, at least about 60%, at least about 70%, at least about 80%, or at least about 90% of the total soluble protein from the transgenic plant.

16. A method of producing a myoglobin protein, wherein the method comprises growing the transgenic plant of claim 1 and isolating the myoglobin protein from the transgenic plant.

17. A method of producing a myoglobin protein in a transgenic plant, wherein the method comprises:
(a) growing the transgenic plant, wherein the transgenic plant comprises at least one chloroplast with one or more recombinant nucleic acid sequences expressing a myoglobin gene encoding the myoglobin protein, and
(b) isolating the myoglobin protein from the transgenic plant.

18. The method of claim 17, wherein the one or more recombinant nucleic acid sequences is integrated into the chloroplast DNA of the transgenic plant, and/or wherein the one or more recombinant nucleic acid sequences is stably integrated into the chloroplast DNA of the transgenic plant.

19. The method of claim 17, wherein the transgenic plant comprises at least about 10 copies, at least about 100 copies, at least about 1,000 copies, at least about 5,000 copies, at least about 10,000 copies, at least about 20,000 copies, at least about 30,000 copies, at least about 40,000 copies, or at least about 50,000 copies of the one or more recombinant nucleic acid sequences.

20. The method of claim 17, wherein the one or more recombinant nucleic acid sequences further comprises:
(a) one or more selectable markers, wherein the one or more selectable markers are optionally removable;
(b) one or more genes encoding one or more enzymes in the heme biosynthesis pathway; and/or
(c) one or more targeting sequences for homologous recombination in the host transgenic plant chloroplast DNA.

21. The method of claim 17, wherein the transgenic plant is: (a) a stable, homoplasmic transformant; or (b) a stable heteroplasmic transformant.

22. The method of claim 17, wherein the myoglobin gene is a bovine myoglobin gene (for example, bison, buffalo, cow, goat, sheep, or yak), an avian myoglobin gene (for example, chicken, duck, goose, guinea fowl, quail, pigeon, or turkey), a suine myoglobin gene (for example, boar or pig), or a fish myoglobin (for example, tuna, salmon, or eel).

23. The method of claim 17, wherein the myoglobin gene is selected from Table 1 and/or wherein the myoglobin gene encodes a myoglobin protein selected from SEQ ID NO's 1-35.

24. The method of claim 17, wherein the myoglobin gene comprises a codon-optimized myoglobin gene, wherein the codon-optimized myoglobin gene is codon-optimized for expression in the transgenic plant.

25. The method of claim 17, wherein the myoglobin gene is operably linked to at least one promoter.

26. The method of claim 17, wherein the transgenic plant is a grass (for example, a barely, a corn, a maize, an oat, a silvergrass, a sugarcane, a rice, a rye, or a wheat), a legume (for example, an alfalfa, a bean, a chickpea, a clover, a lentil, a pea, or a peanut), a nightshade (for example, an eggplant, a pepper, a potato, a tobacco, or a tomato), an aster (for example, a lettuce, a chamomile, an artichoke, an endive, a lavender, a cotton, a sunflower), or an alga, a moss, or a liverwort.

27. The method of claim 26, wherein the transgenic plant is an aster, and the aster is a lettuce plant (i.e., a Lactuca species).

28. The method of claim 26, wherein the transgenic plant is a legume.

29. The method of claim 26, wherein the transgenic plant is a nightshade, and the nightshade is a tobacco plant (i.e., a Nicotiana species)

30. The method of claim 29, wherein the transgenic plant comprises a knock-down or knock-out of one or more genes encoding magnesium chelatase enzymes.

31. The method of claim 17, wherein the myoglobin protein comprises at least about 0.1%, at least about 1.0%, at least about 10%, at least about 20%, at least about 30%, at least about 40%, at least about 50%, at least about 60%, at least about 70%, at least about 80%, or at least about 90% of the total soluble protein from the transgenic plant.

32. A recombinant nucleic acid sequence comprising a myoglobin gene encoding a myoglobin protein, wherein the myoglobin gene is operably linked to at least one promoter.

33. The recombinant nucleic acid sequence of claim 32, wherein the recombinant nucleic acid sequence further comprises:
(a) one or more selectable markers, wherein the one or more selectable markers are optionally removable;
(b) one or more genes encoding one or more enzymes in the heme biosynthesis pathway; and/or
(c) one or more targeting sequences for homologous recombination in the transgenic plant chloroplast DNA.

34. The recombinant nucleic acid sequence of claim 32, wherein the myoglobin gene is a bovine myoglobin gene (for example, bison, buffalo, cow, goat, sheep, or yak), an avian myoglobin gene (for example, chicken, duck, goose, guinea fowl, quail, pigeon, or turkey), a suine myoglobin gene (for example, boar or pig), or a fish myoglobin (for example, tuna, salmon, or eel).

35. The recombinant nucleic acid sequence of claim 32, wherein the myoglobin gene selected from Table 1 and/or wherein the myoglobin gene encodes a myoglobin protein selected from SEQ ID NO's 1-35.

36. The recombinant nucleic acid sequence of claim 32, wherein the myoglobin gene comprises a codon-optimized myoglobin gene, wherein the codon-optimized myoglobin gene is codon-optimized for expression in a transgenic plant.

37. A transgenic plant comprising the recombinant nucleic acid sequence of claim 32.

38. The transgenic plant of claim 37, wherein the recombinant nucleic acid sequence is integrated into the chloroplast DNA of the transgenic plant, and/or wherein the recombinant nucleic acid sequence is stably integrated into the chloroplast DNA of the transgenic plant.

39. The transgenic plant of claim 37, wherein the transgenic plant comprises at least about 10 copies, at least about 100 copies, at least about 1,000 copies, at least about 5,000 copies, at least about 10,000 copies, at least about 20,000 copies, at least about 30,000 copies, at least about 40,000 copies, or at least about 50,000 copies of the one or more recombinant nucleic acid sequences.

40. The transgenic plant of claim 37, wherein the transgenic plant is a grass (for example, a barely, a corn, a maize, an oat, a silvergrass, a sugarcane, a rice, a rye, or a wheat), a legume (for example, an alfalfa, a bean, a chickpea, a clover, a lentil, a pea, or a peanut), a nightshade (for example, an eggplant, a pepper, a potato, a tobacco, or a tomato), an aster (for example, a lettuce, a chamomile, an artichoke, an endive, a lavender, a cotton, a sunflower), or an alga, a moss, or a liverwort.

41. The transgenic plant of claim 40, wherein the transgenic plant is an aster, and the aster is a lettuce plant (i.e., a Lactuca species).

42. The transgenic plant of claim 40, wherein the transgenic plant is a legume.

43. The transgenic plant of claim 40, wherein the transgenic plant is a nightshade, and the nightshade is a tobacco plant (i.e., a Nicotiana species).

44. The transgenic plant of claim 43, wherein the transgenic plant comprises a knock-down or knock-out of one or more genes encoding magnesium chelatase enzymes.

45. The transgenic plant of claim 37, wherein the myoglobin protein comprises at least about 0.1%, at least about 1.0%, at least about 10%, at least about 20%, at least about 30%, at least about 40%, at least about 50%, at least about 60%, at least about 70%, at least about 80%, or at least about 90% of the total soluble protein from the transgenic plant.
